(19) United States
(12) Patent Application Publication
(10) Pub. No.: US 2011/0262571 A1
(43) Pub. Date: Oct. 27, 2011

(54) IRRITABLE BOWEL SYNDROME INHIBITING SUBSTANCE CONTAINING INSOLUBLE DIETARY FIBER FROM SEED OF GRAIN PLANT

(75) Inventors: Osamu Kanauchi, Yokohama-shi (JP); Yutaka Komiyama, Yokohama-shi (JP)

(73) Assignee: KIRIN HOLDINGS KABUSHIKI KAISHA, Chuo-ku, Tokyo (JP)

(21) Appl. No.: 13/140,410

(22) PCT Filed: Dec. 17, 2009
(86) PCT No.: PCT/JP2009/071506

§ 371 Date: Jun. 16, 2011

(30) Foreign Application Priority Data
Dec. 17, 2008 (JP) 2008-321496

(51) Int. Cl. A61K 36/8998; A61P 1/00; A61K 36/899

(52) U.S. Cl. 424/750

(57) ABSTRACT

The present invention provides an irritable bowel syndrome-inhibiting substance containing insoluble dietary fiber, which is obtained by enzymatically treating the seeds of a grain plant or young germinated seeds thereof, and a food or drink or a medicament that contains the substance and has effects of inhibiting irritable bowel syndrome.

### TECHNICAL FIELD

The present invention relates to an irritable bowel syndrome inhibiting substance containing insoluble dietary fiber from plant seeds and young germinated plant seeds thereof and to a method for producing the substance. The present invention also relates to foods or drinks and medicaments comprising the substance as an active ingredient.

### BACKGROUND ART

Irritable bowel syndrome (hereinafter, also referred to as IBS) is characterized by functional hypersensitivity in the intestinal tract. IBS is a syndrome presenting gastrointestinal symptoms such as abnormal laxation (e.g., constipation and diarrhea), abdominal pain, and indefinite complaints as a result of increased motor functions, secretory functions, and the like in the intestinal tract. IBS causes no organic diseases, and psychological factors are also considered to contribute to the expression of IBS symptoms or the exacerbation thereof. ROME III diagnostic criteria are considered to be the worldwide standard criteria for IBS, wherein abdominal symptoms, disease duration, frequency of symptom expression, the relationship with bowel movements, and the like are used as parameters. The precise number of patients is unknown, but the incidence rate in the U.S. ranges from 10% to 20% and it is considered to be around 15% in Japan (Irritable Bowel Syndrome, Seeking Dialogue between Brain and Bowel, Written by Daisuke Sasaki, Nakayama Shoten, Tokyo, Japan).

A therapeutic method is determined depending on the symptoms after exclusion of organic abnormalities. First, dietary and lifestyle instructions are provided, and then a combination of a prokinetic agent, a high molecular weight polymer (colonel), a lactobacillus preparation, a cathartic, and the like is prescribed depending on symptoms. As diet therapies, avoidance of spices containing capsaicin and the like as well as other irritants, ingestion of dietary fibers, and a proposal of an allergen free diet when allergy is suspected are first choices, for example. It has been reported that IBS has a close correlation with changes in enterobacteria because of its features. Probiotics or prebiotics concerning IBS are under clinical evaluation, but the cause of the disease has not yet been determined. Neither conclusive therapeutic methods nor diet therapies exist. Hence, symptomatic therapies are the basic therapies for IBS.

Also from such a perspective, the provision of prebiotics that are well-directed to improve the enteral environment and have few side effects for patients will be good news for improvement of clinical symptoms.

Examples of food ingredients that are materials effective for improvement of the enteral environment include acidic xylo-oligosaccharides obtained by treating wood chips with hemicellulase (JP Patent Publication (Kokai) No. 2007-230998 A), galactomannan and arabinogalactan (JP Patent Publication (Saihyo) No. WO 05/056022), plant-derived proanthocyanidin (JP Patent Publication (Kokai) No. 2007-77122 A), and proteins and insoluble dietary fiber from germinated seeds of Gramineae plants (JP Patent Publication (Kokai) No. 2005-232178 A). Furthermore, examples of reports of cereals being treated with hemicellulase and resulting insoluble residual fractions being imparted with functionality as foods are as follows: an antiulcer agent obtained by treating bran with hemicellulase (JP Patent Publication (Kokai) No. 2006-124370 A); a dietary fiber material for patients with kidney disease obtained by treating wheat bran with hemicellulase (JP Patent Publication (Kokai) No. 06-70720 A (1994)); a method for obtaining a fraction rich in aleurone protein from purified bran comprising a fruit skin fraction and an aleurone fraction obtained from cereal bran by enzymatic treatment and wet pulverization; and use of the fractions in foods and feeds (JP Patent Publication (Kohyo) No. 2004-520058 A). However, there are no examples of reports concerning the effects of inhibiting IBS.

### DISCLOSURE OF THE INVENTION

An object of the present invention is to provide a safe material having inhibitory effects on IBS. Another object of the present invention is to provide a food or drink for inhibiting IBS and an IBS inhibiting agent comprising such a material.

As a result of intensive studies for solving such problems, the present inventors have now found that an insoluble fraction containing an insoluble dietary fiber, which is prepared by removing starch from seeds of a grain plant and preferably a Gramineous plant and then subjecting the remaining fruit skin/seed coat, etc., to hemicellulase treatment, has the effect of inhibiting IBS. Thus, the present inventors have now completed the present invention. The gist of the present invention is as follows.

(1) A substance for inhibiting irritable bowel syndrome, containing insoluble dietary fiber from the seeds of a grain plant, wherein the substance is produced by treatment comprising the steps of:
(a) preparing a raw material by pulverizing or polishing the seeds of a grain plant and then recovering the resultant outer fraction of the seeds; (b) subjecting the raw material to starch removal treatment to prepare a starch-free fraction; (c) enzymatically treating the fraction prepared in step (b) with an enzyme having hemicellulase activity; and (d) recovering an insoluble fraction from the enzymatically treated solution.
(2) The substance according to (1) above, wherein the grain plant is a Gramineae plant.
(3) The substance according to (2) above, wherein the Gramineae plant is rice, barley, rye, or wheat.
(4) The substance according to (1) above, wherein in step (b), the starch removal treatment is performed by enzymatic treatment using amylase or glucoamylase.
(5) The substance according to (1) above, wherein in step (b), the starch removal treatment is performed by heat gelatinization treatment.
(6) The substance according to (1) above, wherein in step (b), the starch removal treatment is performed by physical destruction treatment.
(7) The substance according to (6) above, wherein the physical destruction treatment is performed using a homogenizer.
(8) The substance according to (1) above, wherein in step (b), the starch-free fraction is further subjected to press-peeling treatment.
(9) The substance according to (1) above, wherein the raw material to be subjected to the starch removal treatment is rice bran, wheat malt, or barley malt.
(10) The substance according to (1) above, wherein the raw material to be subjected to the starch removal treatment is defatted rice bran.
(11) The substance according to (8) above, wherein the raw material to be subjected to the starch removal treatment is wheat bran or polished-barley residue.
(12) The substance according to (8) above, wherein the starch-free fraction is brewer's grain.
(13) The substance according to any one of (1) to (12) above, wherein the enzyme having hemicellulase activity is xylanase.
(14) The substance according to any one of (1) to (13) above, wherein, in step (c), the enzyme is used in combination with protease.
(15) The substance according to any one of (1) to (14) above, wherein step (c) is further followed by defatting treatment.
(16) The substance according to any one of (1) to (15) above, wherein the insoluble fraction in step (d) comprises a fraction having a grain size that allows it to substantially pass through a 5- to 25-mesh ASTM standard sieve (American Society for Testing and Materials) but not to pass through a 500-mesh ASTM standard sieve.
(17) The substance according to (16) above, wherein the insoluble fraction in step (d) comprises a fraction having a grain size that does not substantially allow it to pass through a 200-mesh ASTM standard sieve.
(18) The substance according to any one of (1) to (17) above, wherein the protein content is 20 wt % or less and the dietary fiber content is 55 wt % or more.
(19) The substance according to any one of (1) to (18) above, wherein an aleurone layer is partially or completely removed from the insoluble dietary fiber.
(20) A substance for inhibiting irritable bowel syndrome containing an insoluble dietary fiber from the seeds of a grain plant, having the following properties:
(i) the substance comprises a fraction having a grain size that allows it to substantially pass through a 5- to 25-mesh ASTM standard sieve (American Society for Testing and Materials) but not to pass through a 500-mesh ASTM standard sieve;
(ii) the protein content is 20 wt % or less and the dietary fiber content is 55 wt % or more; and
(iii) the aleurone layer of insoluble dietary fiber is partially or completely removed.
(21) The substance according to (20) above, comprising a fraction that does not substantially pass through a 200-mesh ASTM standard sieve.
(22) A food or drink for inhibiting irritable bowel syndrome, containing the substance according to any one of (1) to (21) above as an active ingredient.
(23) A pharmaceutical composition for inhibiting irritable bowel syndrome, containing the substance according to any one of (1) to (21) above as an active ingredient.
(24) Use of the substance according to any one of (1) to (21) above for producing a food or drink or medicament for inhibiting irritable bowel syndrome.
(25) A method for producing a substance for inhibiting irritable bowel syndrome containing insoluble dietary fiber from the seeds of a grain food, comprising the following steps of:
(a) preparing a raw material by pulverizing or polishing the seeds of a grain plant and then recovering the resultant outer fraction of the seeds;
(b) subjecting the raw material to starch removal treatment to prepare a starch-free fraction;
(c) enzymatically treating the fraction prepared in step (b) with an enzyme having hemicellulase activity; and
(d) recovering an insoluble fraction from the enzymatically treated solution.
(26) A method for producing the substance according to (20) above, comprising the following steps of:
(a1) preparing a raw material by pulverizing or polishing the seeds of a grain plant and then recovering the resultant outer fraction of the seeds;
(b1) subjecting the raw material to starch removal treatment via physical destruction to prepare a starch-free fraction; and
(c1) recovering, from the fraction prepared in step (b1), an insoluble fraction that passes through a 5- to 25-mesh ASTM standard sieve (American Society for Testing and Materials), but does not pass through a 500-mesh ASTM standard sieve.
(27) The substance for inhibiting irritable bowel syndrome according to (20) or (21) above, which is produced by the method according to (25) or (26) above.

The description comprises the contents described in the description and/or drawings of Japanese Patent Application No. 2008-321496 from which the present application claims the priority.

### BRIEF DESCRIPTION OF THE DRAWINGS

FIG. 1 shows the photograph of an aleurone layer portion contained in Preparation Sample 1.

FIG. 2 shows the photograph of an aleurone layer portion contained in Preparation Sample 2.

FIG. 3 shows the photograph of an aleurone layer portion contained in Preparation Sample 3.

FIG. 4 shows the photograph of an aleurone layer portion after starch alone has been removed with cold water without reaction with amylase at 65° C. in Preparation Sample 3.

FIG. 5 shows the photograph of an aleurone layer portion contained in Preparation Sample 4.

FIG. 6 shows the photograph of an aleurone layer portion contained in Preparation Sample 5.

FIG. 7 shows the photograph of an aleurone layer portion contained in Preparation Sample 6.

FIG. 8 shows the photograph of an aleurone layer portion contained in Preparation Sample 14.

FIG. 9 shows the photograph of an aleurone layer portion contained in Preparation Sample 15.

FIG. 10 shows the comparison of fecal outputs between a restraint stress model experimental group and a comparative group. In FIG. 10, asterisk indicates significant difference (P<0.05).

FIG. 11 shows the comparison of CRD (colorectal distension) pressures, which represent a pain threshold and which were obtained by the barostat method, between a restraint stress model experimental group and a comparative group. In FIG. 11, asterisk indicates significant difference (P<0.05). The pain threshold was determined based on AWR (abdorminal withdrawal reflex; Al-Chaer E D, Kawasaki M, Pasricha P J., Gastroenterology. 2000 November; 119 (5): 1276-85).

FIG. 12 shows the comparison of serotonin contents in large intestinal mucosa between a restraint stress model experimental group and a comparative group. In FIG. 12, asterisk indicates significant difference (P<0.05).

FIG. 13 shows the photograph of an aleurone layer portion contained in Preparation Sample 21.

FIG. 14 shows the photograph of an aleurone layer portion contained in Preparation Sample 22.

FIG. 15 shows the photograph of an aleurone layer portion contained in Preparation Sample 23.

FIG. 16 shows recovery rates when Preparation Samples 21 and 22 were degraded again with an enzyme.

FIG. 17 shows the amount of free sugar when Preparation Samples 21 and 22 were degraded again with an enzyme.

FIG. 18 shows the effect of improving clinical findings on human patients with irritable bowel syndrome who ingested the insoluble dietary fiber-containing substance prepared according to Preparation Example 16.

### BEST MODE FOR CARRYING OUT THE INVENTION

The IBS-inhibiting substance of the present invention is based on the finding that an insoluble fraction containing insoluble dietary fiber (hereinafter, also referred to as “an insoluble dietary fiber-containing substance”) from the seeds of a grain plant has the effect of inhibiting IBS.

The insoluble dietary fiber-containing substance, the method for producing the same, and the foods or drinks and medicaments for inhibiting IBS comprising the substance as an active ingredient of the present invention will be described below.

The insoluble dietary fiber-containing substance of the present invention is characterized by containing insoluble dietary fiber from the seeds of a grain plant and preferably a Gramineae plant.

Examples of the Gramineae plant that can be used in the present invention include all plants classified as Gramineae plants listed in the plant classification table. Specific examples include, but are not limited to, rice, barley, wheat, rye, foxtail millet, Japanese millet, and corn. Of them, rice, barley, rye, and wheat are preferably used.

The insoluble dietary fiber-containing substance of the present invention can be produced by removing, e.g., starch from the seeds of a grain plant and preferably a Gramineae plant, as a raw material, subjecting the resultant starch-free fraction to enzymatic treatment, and then recovering the resultant insoluble fraction.

Specifically, the insoluble dietary fiber-containing substance can be produced by treatment comprising the steps of: (a) preparing a raw material by pulverizing or polishing the seeds of a grain plant and then recovering the resultant outer fraction of the seeds; (b) subjecting the raw material to starch removal treatment to prepare a starch-free fraction; (c) enzymatically treating the fraction prepared in step (b) with an enzyme having hemicellulase activity; and (d) recovering an insoluble fraction from the enzymatically treated solution.

Step (a) involves pulverizing and/or polishing the seeds of a grain plant to obtain a raw material for preparing the insoluble dietary fiber-containing substance of the present invention. Pulverization can be performed using a roll mill, a disk mill, a hammer mill, or the like. Furthermore, the term “polishing” means to remove and separate fruit skin, seed coats, germs, etc. from the seeds of a grain plant. For example, this can be performed using a machine that works based on the principle that the surface is scraped off in the case of a rice mill or a stone mill. When polishing is performed, a seed outer fraction including fruit skin, seed coats, and germs produced by such treatment can be used as a raw material in step (b). This step may involve threshing in addition to pulverization and/or polishing of the seeds of a grain plant when the seeds have husks. Furthermore, the seeds of a grain plant to be used herein may be either germinated or ungerminated.

Step (a) can be omitted when a material treated in advance in a manner substantially equivalent to that in step (a) is used as a raw material. Examples of such a material include, but are not limited to, bran that is secondarily produced when wheat powder is produced and rice bran that is secondarily produced after rice polishing. Use of such a material is preferred since most starch has already been removed from the seeds of a grain plant and in view of simplification of the steps. When rice bran is used as a raw material, it is preferred to use defatted rice bran produced after defatting treatment of rice bran. This is because the defatted rice bran is a material cheaper than rice bran and has an excellent flavor. In addition, compared to a case where the same amount of rice bran is used, a larger amount of the insoluble dietary fiber-containing substance can be advantageously obtained.

Step (b) involves removing starch from the raw material prepared in step (a) to prepare a starch-free fraction. Any method may be employed for the starch removal treatment as long as it can remove starch from the raw material. For example, starch removal treatment can be performed by enzymatic starch degradation treatment and then heat gelatinization followed by sieving, or by physical destruction.

When the starch removal treatment is performed by saccharification treatment using an enzyme, starch can be removed using amylase or glucoamylase, but the example is not limited thereto. Examples of amylase and glucoamylase that can be used in the present invention include, but are not limited to, a commercially available enzyme preparation such as Orientase (trade name, HBI Inc.), Kokugen (trade name, Daiwa Fine Chemicals Co., Ltd.), Sumizyme (trade name, Shinnihon Chemicals Co., Ltd.), Termamyl (trade name, Novozymes), and Gluczyme (trade name, Amano Enzyme Inc.), and amylases and glucoamylases produced by microorganisms belonging to the genus Trichoderma, Thermomyces, Aureobasidium, Streptomyces, Aspergillus, Clostridium, Bacillus, Thermotogae, Thermoascus, Caldocellum, Thermomonospora, Humicola, Rhizopus, or Penicillium, for example. The aforementioned enzymes usable in the present invention may be in any forms such as a purified enzyme, a crude enzyme, and an immobilized enzyme, as long as they maintain enzymatic activity. An immobilized enzyme is an enzyme bound to a carrier such as a polymer, a polysaccharide, and an inorganic material. Examples of a crude enzyme include an enzyme-containing extract from an enzyme-containing microorganism and a processed product such as a dry product. Furthermore, saccharification treatment is performed generally at a temperature between 10° C. and 90° C., and preferably between 30° C. and 60° C., at a pH between 3 and 10, and preferably between 5 and 7. It is preferable to perform saccharification at a temperature near an optimal temperature or at a pH near an optimal pH for amylase and glucoamylase to be used. The enzymatic treatment can last for a period of from 30 minutes to overnight; for example, 3 hours. Note that the amount of enzyme to be added can be determined by persons skilled in the art depending on the amount of the raw material prepared.

Heat gelatinization can be performed using a phenomenon such that when starch is suspended in water and heated, starch particles absorb water, gradually swell, finally collapse, and thus are dissolved. Thereafter, the gelatinized starch can be removed by solid-liquid separation using a sieve.

Starch removal treatment involving physical destruction can be performed using a homogenizer, a high-speed mixer, a homo mixer, a stirrer (homogenizer), or the like. Not only starch but also a portion of an aleurone layer can be removed by the starch removal treatment involving physical destruction using these examples.

When germinated seeds are used as the seeds of a grain plant, the starch removal treatment can be performed simply by heating without external addition of amylase or glucoamylase. This is because germinated seeds themselves produce amylase in this case.

In the production method of the present invention, a material treated in a manner substantially equivalent to that in steps (a) and (b) may be used as a starch-free fraction in the following steps. Examples of such a material include, but are not limited to, brewer's grain; that is, barley malt generated when beer is produced. It is preferred to use such a material since most starch has already been removed and in view of simplification of the steps.

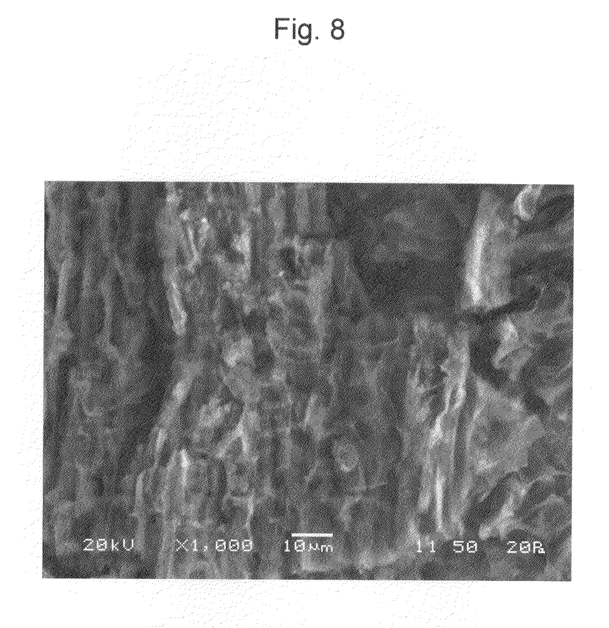

The starch-free fraction prepared by step (b) may contain not only major ingredients such as an aleurone layer, fruit skin, and a seed coat, but also a husk, a germ, another protein, and a lipid, etc. The plant tissues of malt, such as an aleurone layer, fruit skin, and a seed coat are shown in Barley: Chemistry and Technology edited by Alexander W. MacGregor et al., p. 46, FIG. 8. Furthermore, these are commonly present in the seeds of Gramineae plants as shown in Journal of Cereal Science 36 (2002) 261-284.

Step (b) may further involve press-peeling treatment of a starch-free fraction. The press-peeling treatment refers to treatment involving causing a multi-layer composition to pass through an appropriate gap between two roll mills rotating at slightly different rotational speeds, so as to apply force in parallel to the pressed surface, thereby peeling a portion of the layer. In the press-peeling treatment, any pulverizer can be used as long as it has a structure of applying compression force to the raw material to be treated. For example, in the press-peeling treatment, in addition to the roll mills differing in rotational speed, a mortar-form pressurizing treatment apparatus having a gap and a controlled rotational speed can be used. Particularly, a roll mill is desirably used. At this time, the gap between the rolls ranges from 0.15 mm to 0.01 mm and preferably ranges from 0.08 mm to 0.02 mm. The size is selected such that physical pulverization can be efficiently applied to a multi-layered composition to be peeled. In this manner, the aleurone layer contained in a starch-free fraction is exposed, so that removal treatment of an aleurone layer via enzymatic treatment with hemicellulase in step (c) can be more efficiently performed. Persons skilled in the art can appropriately select the presence or the absence of the press-peeling treatment depending on the raw material to be used and the types or conditions of a starch-free fraction. For example, it can be determined that press-peeling treatment is performed when wheat bran, a polished-barley residue, and brewer's grain are used as a raw material and a starch-free fraction; but press-peeling treatment is not performed when rice bran, defatted rice bran, wheat malt, and barley malt are used as a raw material and a starch-free fraction. It is also suggested that when germinated seeds are used as the seeds of a grain plant, such press-peeling treatment can be omitted.

Step (b) may further involve sieving treatment of a starch-free fraction which is performed in the presence of water. Sieving treatment is performed in order to remove husks and other nontarget substances contained in large amounts in a starch-free fraction, thereby roughly separating a fraction (hereinafter also referred to as a fruit skin/seed coat fraction) containing an aleurone layer, fruit skin, and a seed coat as major ingredients. The sieving treatment can be performed by, for example, allowing a starch-free fraction to pass through a mesh having an appropriate mesh size and removing the fraction remaining on the mesh. The mesh size of the sieve to be used in the sieving treatment may vary depending on the raw material and starch-free fraction to be used herein. Typically, 5- to 25-mesh (e.g., 12-mesh, 16-mesh, or 20-mesh) ASTM standard sieves (American Society for Testing and Materials) can be used. This treatment is preferred in view of handling in the subsequent operations since a large amount of nontarget substances can be removed.

In step (b), the press-peeling treatment and the sieving treatment are preferably used in combination. In this case, preferably, the thus combined treatment is performed 2 to 5 times repeatedly. This is because a fraction containing an aleurone layer, fruit skin, and a seed coat as major ingredients can be more efficiently obtained.

As mentioned above, whether or not pretreatment such as press-peeling treatment is performed can be determined depending on the types of and the germinated state of the seeds of a grain plant to be used herein and the types of raw material, for example. Specifically, steps (a) and (b) above can be defined as pretreatment steps for more efficiently performing degradation of the aleurone layer via enzymatic treatment with hemicellulase in step (c). Therefore, regarding the starch-free fraction to be subjected to step (c), it is preferable to confirm before step (c) whether or not pretreatment in steps (a) and (b) has been optimally performed and then to add or repeat the pretreatment such as press-peeling treatment as necessary. At this time, a tissue of the starch-free fraction prepared in steps (a) and (b), for example, is microscopically observed or analyzed using an iodine starch reaction. Hence, it can be determined if pretreatment has been optimally performed.

Step (c) involves removing the aleurone layer contained in a starch-free fraction or a fruit skin/seed coat fraction prepared in step (b) as described above. This step involves enzymatic treatment of a starch-free fraction or a fruit skin/seed coat fraction with an enzyme having hemicellulase enzymatic activity. Examples of such an enzyme having hemicellulase enzymatic activity that can be used in this step include, but are not limited to, β-glucosidase, cellulase, xylosidase, xylanase, mannosidase, mannanase, arabinosidase, arabanase, pectinase, and glucanase. Particularly, a xylan-degrading enzyme including xylanase is preferably used. Specific examples of hemicellulase that can be used in the present invention include, but are not limited to, commercially available enzyme preparations such as Cellulosin (trade name, HBI Inc.), Multifect 720 (trade name, Genencor Kyowa), Sumizyme (trade name, Shinnihon Chemicals Co., Ltd.), Pentopan (trade name, Novozymes) and hemicellulase “Amano” 90 (trade name, Amano Enzyme Inc.); and xylanase produced by microorganisms belonging to the genus Trichoderma, Thermomyces, Aureobasidium, Streptomyces, Aspergillus, Clostridium, Bacillus, Thermotogae, Thermoascus, Caldocellum, Thermomonospora, Humicola, Rhizopus, or Penicillium, for example.

The aforementioned enzymes to be used in the present invention can be used in any forms such as a purified enzyme, a crude enzyme, and an immobilized enzyme as long as the enzymes maintain their enzymatic activity. The immobilized enzyme is an enzyme bound to a carrier such as a polymer, a polysaccharide, and an inorganic material. Examples of a crude enzyme include an enzyme-containing extract from an enzyme-containing microorganism and a processed product such as a dry product. Enzymatic treatment with hemicellulase is generally performed at a temperature between 10° C. to 90° C. and preferably between 30° C. and 60° C. at a pH between 3 and 10 and preferably between 5 and 7; however, it is preferred that the enzymatic treatment is performed at a temperature near the optimal temperature or at a pH near the optimal pH of a hemicellulase enzyme to be used herein. Furthermore, the time for enzymatic treatment can be 30 minutes to overnight. Note that the amount of an enzyme to be added can be determined by persons skilled in the art depending on the amount of a starch-free fraction or a fruit skin/seed coat fraction prepared. It is preferable to visually confirm using e.g., a fluorescent microscope or an electron microscope that the aleurone layer in a starch-free fraction or a fruit skin/seed coat fraction has been partially or completely removed after the enzymatic treatment with hemicellulase. Confirmation can be made using a method generally employed by persons skilled in the art as an analytical method using a fluorescent microscope, such as that described in “PLANT MICROTECHNIQUE AND MICROSCOPY” written by STEVEN E. RUZEN, Chapter 7 (pp. 87-119). Furthermore, confirmation can be made by an electron-microscopic analysis method such as a method generally used by persons skilled in the art.

In the enzymatic treatment of step (c), protease treatment is also preferably used in combination. Thus, the amount of an enzyme having hemicellulase enzymatic activity to be used for degrading an aleurone layer can be reduced to an amount ⅕ to 1/10 the amount of the enzyme that is used independently. This is advantageous in terms of cost.

Step (c) preferably involves defatting treatment that is performed after hemicellulase treatment. This can improve the taste and the quality of the insoluble dietary fiber-containing substance of the present invention. The defatting method is not particularly limited and can be performed by defatting treatment using ethanol, acetone, hexane, or the like. These techniques are obvious to persons skilled in the art. It should be noted that when defatted rice bran is used as a raw material to be subjected to the starch removal treatment, the aforementioned benefit can be obtained without such defatting treatment.

Step (d) is a step of recovering an insoluble fraction from the enzymatically treated solution in step (c). Preferably, an insoluble fraction to be recovered in this step passes through a 5- to 25-mesh ASTM standard sieve, but does not pass through a 500-mesh ASTM standard sieve.

Specifically, the sieving treatment can be performed by a two-step system. That is, a solution treated with hemicellulase is sieved using a first mesh, the fraction having passed through the sieve is sieved using a second mesh that has a smaller mesh size than the first mesh, and then the insoluble dietary fiber-rich insoluble fraction remaining on the sieve (more specifically, “insoluble dietary fiber-containing substance”) is recovered. The mesh sizes of the first and second meshes to be used in the sieving treatment are not particularly limited, as long as the fraction having the aforementioned particle size can be recovered with the use of the combination of the two meshes. For example, as the first mesh, a 5- to 25-mesh ASTM standard sieve such as a 12-mesh, a 16-mesh, or preferably a 20- to 25-mesh sieve, is selected. As the second mesh, a 50- to 500-mesh ASTM standard sieve can be selected. A desired fraction in the enzymatically treated solution can be obtained by the sieving treatment.

Alternatively, an insoluble fraction to be recovered in step (d) can also be recovered as a fraction that does not pass through a 200-mesh ASTM standard sieve with the use of a 50- to 200-mesh ASTM standard sieve as the second mesh.

Note that, in step (b), when the fruit skin/seed coat fraction is roughly fractioned in advance, step (d) is not necessarily performed by the two-step system. The fraction remaining on the sieve as a result of the sieving treatment using the second mesh may be recovered.

The insoluble fraction obtained in step (d) is generally dried by lyophilization or the like, and can be used as the insoluble dietary fiber-containing substance of the present invention preferably without further processing such as pulverization into fine particles.

The thus produced insoluble dietary fiber-containing substance of the present invention comprises a fraction that passes through preferably substantially a 5- to 25-mesh ASTM standard sieve, but does not pass through a 500-mesh ASTM standard sieve based on the sieving treatment of step (d). The term “substantially” as used herein is used to mean that an insoluble dietary fiber-containing substance contaminated with a small amount of an ingredient outside the above grain size range falls within the scope of the invention. Accordingly, the insoluble dietary fiber-containing substance of the present invention comprising a fraction that substantially passes through a 5- to 25-mesh ASTM standard sieve, but does not pass through a 500-mesh ASTM standard sieve contains a fraction with the aforementioned grain size in an amount of at least 90%, preferably 91%, 92%, 93%, 94%, 95%, 96%, 97%, 98% or 99%, and most preferably 100%.

Furthermore, the insoluble dietary fiber-containing substance of the present invention obtained by the above-mentioned production methods has a property such that the protein content is preferably 20 wt % or less (e.g., 15 wt % or less and 10 wt % or less) and the insoluble dietary fiber content is preferably 55 wt % or more, more preferably 60 wt % or more, and further more preferably 70 wt % or more (e.g., 75 wt % or more and 80 wt % or more). The “protein content” as used herein is found on the basis of a numerical value provided by multiplying the nitrogen content found using the Kjeldahl method by 6.25 representing a protein conversion coefficient. The dietary fiber content is found based on the AOAC method.

Furthermore, in the insoluble dietary fiber contained in the insoluble dietary fiber-containing substance of the present invention, the aleurone layer has been partially or completely removed. The term “partially” as used herein means that 70% or more, preferably 80% or more, more preferably 90% or more, even more preferably 95% or more, and most preferably 99% or more of the aleurone layer of the insoluble dietary fiber has been removed. The aleurone layer of the insoluble dietary fiber preferably has been completely removed.

Partial or complete removal of the aleurone layer from the insoluble dietary fiber can be visually confirmed by a fluorescent microscope, an electron microscope, or the like, as mentioned above. Alternatively, the same can be confirmed by, for example, subjecting the insoluble dietary fiber-containing substance of the present invention to enzymatic treatment with hemicellulase and evaluating the recovery rate after the treatment. Since the insoluble dietary fiber-containing substance of the present invention is a fraction from which most of the aleurone layer has been removed as described above, even if the fraction is subjected again to enzymatic treatment with hemicellulase, the recovery rate after the treatment is extremely high. Specifically, the insoluble dietary fiber-containing substance of the present invention can be characterized by having a recovery rate of at least about 70% and preferably at least about 80%, when it is subjected again to overnight enzymatic treatment with hemicellulase in a 50 mM acetate buffer (pH 4.5, 50° C.) containing a 4.0% hemicellulase preparation (Sumizyme NX, Shinnihon Chemicals Co., Ltd.). Note that the term “recovery rate” as used herein refers to a relative value; that is, the weight of a substance (obtained by lyophilization after enzymatic treatment (4.0% hemicellulase, pH 4.5, 50° C., overnight) performed again for the insoluble dietary fiber-containing substance) with respect to the weight of the unlyophilized insoluble dietary fiber-containing substance as 100%.

The insoluble dietary fiber-containing substance of the present invention has other properties. For example, swelling ability can be mentioned. The term “swelling ability” as used herein refers to the property such that water molecules are incorporated into the insoluble dietary fiber-containing substance (between the plant tissues) and the particles of the substance are swollen and increase in size. That the insoluble dietary fiber-containing substance of the present invention has swelling ability can be confirmed by weighing a predetermined amount of the insoluble dietary fiber-containing substance into a volumetric graduated cylinder, adding a predetermined volume of water to the cylinder, allowing it to stand still for a predetermined time, and then determining the degree of swelling by graduation of the graduated cylinder, for example. The swelling ability of the insoluble dietary fiber-containing substance of the present invention can be characterized by swelling to a volume of 20 ml-35 ml per gram.

Production of the insoluble dietary fiber-containing substance of the present invention will be more specifically described below when brewer's grain is used as a starch-free fraction and wheat bran or rice bran is used as a raw material.

When the insoluble dietary fiber-containing substance of the present invention is produced, it is economically preferable to use brewer's grain, i.e. the barley malt generated during beer production, bran secondarily produced when wheat powder is produced, rice bran secondarily produced after rice polishing, or the like, as described above.

When brewer's grain is used for producing the insoluble dietary fiber-containing substance of the present invention, the insoluble dietary fiber-containing substance of the present invention can be obtained by a method specifically described, for example, in JP Patent Publication (Kokoku) No. 4-31666 B (1992). More specifically, wet brewer's grain is pulverized under pressure and the resultant pressurized and pulverized material is sieved in the presence of water to prepare a fraction containing fruit skin/seed coat and an aleurone layer in large amounts. The fraction may be treated with hemicellulase.

More specifically, to remove husks intrinsic to barley, wet brewer's grain is subjected to press-peeling treatment. In the press-peeling treatment for brewer's grain, any pulverizer can be used as long as it has a structure of applying compression force to the raw material to be treated; however, particularly, a roll mill is desirably used. The gap between rolls ranges from 0.15 mm to 0.01 mm, preferably ranges from 0.08 mm to 0.02 mm; however, the size is selected such that physical destruction of the multilayered material to be peeled off can be efficiently performed. When brewer's grain is subjected to press-peeling treatment, it is desirable to adjust the moisture content of the brewer's grain to 65% or more. Next, the thus obtained press-peeled material is subjected to sieving treatment in the presence of water. Through the sieving treatment, a husk fraction remains on the sieve, whereas an aleurone layer-rich fruit skin/seed coat fraction passes through the sieve. The dimension of the sieve mesh ranges from 5- to 20-mesh and preferably ranges from 16- to 20-mesh of an ASTM standard sieve. Through the treatment above, a fraction containing husks intrinsic to barley in a large amount can be removed. Furthermore, to efficiently obtain a fruit skin/seed coat fraction, sieving is further performed using a sieve with a smaller mesh size than that of the previously used sieve. At this time, the fruit skin/seed coat fraction remains on the sieve and thus the fruit skin/seed coat fraction can be prepared. The dimension of the latter sieve mesh is determined to be 50- to 200-mesh of an ASTM standard sieve. The press-peeling treatment and the sieving treatment are preferably repeatedly performed 2 to 5 times. The fruit skin/seed coat fraction obtained as mentioned above is subjected to hemicellulase treatment and is subsequently sieved again using a 50- to 200-mesh ASTM standard sieve to remove substances degraded with hemicellulase. The insoluble fraction remaining on the sieve is a target insoluble dietary fiber-containing substance. The thus obtained insoluble dietary fiber-containing substance is usually dried and then used. An example of the drying method is, but is not particularly limited to, a lyophilization method.

When wheat bran is used for producing the insoluble dietary fiber-containing substance of the present invention, first dry wheat bran is suspended in a solution containing amylase or glucoamylase to remove starch, thereby degrading starch. After amylase reaction is performed for a long time (for example, 30 minutes to overnight), press-peeling treatment is performed using, for example, a roll mill, in order to efficiently perform enzymatic treatment with hemicellulase. The gap between rolls ranges from 0.15 mm to 0.01 mm and preferably ranges from 0.08 mm to 0.02 mm; however, the size is selected such that physical destruction of the multilayered material to be peeled off can be efficiently performed. Next, sieving is performed using a 50- to 200-mesh ASTM standard sieve and preferably using a 200-mesh ASTM standard sieve to remove amylase-degraded products, and a fruit skin/seed coat fraction remaining on the sieve is recovered. Next, this fraction is suspended in a solution containing hemicellulase and reacted for a long time (for example, 30 minutes to overnight). After completion of the reaction, sieving is similarly performed using a 50- to 200-mesh and preferably, a 200-mesh ASTM standard sieve, so as to remove hemicellulase-degraded products and to recover an insoluble fraction remaining on the sieve. Thus, the insoluble dietary fiber-containing substance of the present invention can be obtained. The thus obtained insoluble dietary fiber-containing substance is usually dried and then used. A specific example of the drying method is, but is not limited to, a lyophilization method.

When rice bran is used for producing the insoluble dietary fiber-containing substance of the present invention, first, dry rice bran is suspended in a solution containing amylase or glucoamylase to remove starch, thereby degrading starch. After an amylase reaction is performed for a long time (for example, 30 minutes to overnight), sieving is performed using a 50- to 200-mesh and preferably a 200-mesh ASTM standard sieve so as to remove amylase-degraded products and a fruit skin/seed coat fraction remaining on the sieve is recovered. Next, the recovered fraction is suspended in a solution containing hemicellulase and reacted for a long time (for example, 30 minutes to overnight). After completion of the reaction, sieving is similarly performed using a 50- to 200-mesh and preferably a 200-mesh ASTM standard sieve, so as to remove hemicellulase-degraded products and the fraction remaining on the sieve is recovered. Thus, the insoluble dietary fiber-containing substance of the present invention can be obtained. The thus obtained insoluble dietary fiber-containing substance is usually dried and then used. An example of the drying method is, but is not limited to, a lyophilization method.

The insoluble dietary fiber-containing substance of the present invention and the method for producing the same are as described above. However, the production method of the insoluble dietary fiber-containing substance of the present invention is not limited to the above-described methods. Specifically, as long as an insoluble dietary fiber-containing substance having the following properties (i) to (iii) can be produced from seeds of a grain plant, other production methods may be employed. The properties are: (i) the insoluble dietary fiber-containing substance substantially comprises a fraction having a grain size that passes through a 5- to 25-mesh ASTM standard sieve, but does not pass through a 500-mesh ASTM standard sieve; (ii) the insoluble dietary fiber-containing substance has a protein content of 20 wt % or less and a dietary fiber content of 55 wt % or more; and (iii) the aleurone layer of the insoluble dietary fiber is partially or completely removed.

An example of an alternative method for producing the insoluble dietary fiber-containing substance of the present invention having the aforementioned properties is a method that involves the following steps of: (a1) preparing a raw material by pulverizing or polishing the seeds of a grain plant and then recovering the resultant outer fraction of the seeds; (b1) subjecting the raw material to starch removal treatment by physical destruction; and (c1) recovering an insoluble fraction that substantially passes through a 5- to 25-mesh ASTM standard sieve but does not pass through a 500-mesh ASTM standard sieve from the fraction prepared in the step (b1).

This method can also be performed similarly to the above method, except that the starch removal treatment involving physical destruction is performed and the method involves no enzymatic treatment step with an enzyme having hemicellulase activity.

In such a method, the starch removal treatment involving physical destruction can be performed by treatment using a homogenizer, a high-speed mixer, a homo mixer, or a stirrer, for example. In this manner, the aleurone layer can be removed simultaneously with starch removal treatment.

The starch removal treatment is preferably performed a plurality of times, for example, 2 to 5 times, in combination with sieving treatment described in the above method in step (b). In this manner, the starch removal treatment and the accompanying aleurone layer removal can be more efficiently performed.

The starch removal treatment may be performed in combination with other starch removal treatments excluding physical destruction, such as enzymatic starch degradation treatment, heat gelatinization, and gelatinization followed by sieving.

The insoluble dietary fiber-containing substance of the present invention produced as described above is characterized in that it is ingested and/or administered in order to inhibit IBS. IBS is also referred to as irritable large bowel syndrome, which is a generic name for diseases mainly resulting from abnormalities in large bowel motility and visceral sensation. In the case of IBS, no visible abnormalities are observed, such as inflammation or ulcer, but abnormalities in laxation, such as diarrhea or constipation, and symptoms such as lower abdominal bloating because of excessive gas are observed. IBS can be divided into four types: unstable-type IBS, chronic diarrheal-type IBS, secretory-type IBS, and gas-type IBS, depending on how the symptoms appear. The insoluble dietary fiber-containing substance of the present invention may be used for any type of IBS. The expression “ . . . inhibit IBS” as used herein refers to the improvement or prevention of one or more IBS-related symptoms, and to improvement or maintenance of one or more IBS-related parameter values. Examples of the above symptoms include constipation, diarrhea, abdominal pain, abdominal bloating, an urgent need to defecate, and a sense of incomplete evacuation. The expression “ . . . improve symptoms” refers to improvement in the severity, disease duration, and expression frequency of these symptoms. Also, examples of the above parameter values include serotonin content in large intestinal mucosa, pain threshold in the large bowel, and fecal output upon exposure to stress. The expression “ . . . improve parameter values” refers to recovery resulting in parameter values representing normal levels through exposure or lack of exposure to stress.

The insoluble dietary fiber-containing substance of the present invention can inhibit IBS through direct ingestion and/or administration of the substance as an IBS-inhibiting substance or as an active ingredient in a food, drink, or medicament. Also, daily ingestion and/or administration of the substance can prevent IBS. For this purpose, it is desirable to ingest and/or administer 1 g or more, 2 g or more, 3 g or more, 4 g or more, 5 g or more, 6 g or more, 7 g or more, 8 g or more, or 9 g or more, and preferably 10 g or more, 12 g or more, 14 g or more, 16 g or more, 18 g or more, 20 g or more, or 30 g or more of the insoluble dietary fiber-containing substance of the present invention per day.

The insoluble dietary fiber-containing substance of the present invention is an ingredient obtained by treating the seeds of a grain plant with a food enzyme, so that it can become a part of daily dietary life. Furthermore, the administration or ingestion may take place before a meal, between meals, or after a meal.

When the insoluble dietary fiber-containing substance of the present invention is contained in a food or drink and the food or drink for inhibiting IBS is provided, the substance can be processed into any form of foods. Examples of the food or drink, in which the insoluble dietary fiber-containing substance of the present invention can be contained, include foods or drinks including natural products and processed foods thereof. Furthermore, the insoluble dietary fiber-containing substance of the present invention can be contained in an amount ranging from 0.1 g to 90 g and preferably ranging from about 1 g to 50 g per 100 g of a food or drink, but the amount of the substance differs depending on the form of a food or drink.

Examples of the food or drink include, but are not limited to, functional foods such as a tablet food, a powder food, a granular food, a capsule food, and a jelly food, processed cereal products such as bread, confectionaries, cookies, and biscuits, dairy products such as milk, yogurt, and ice cream, beverages such as a carbonated drink, a soft drink, a fruit juice-containing beverage, and a medicinal drink, prepared foods, and processed foods.

When the insoluble dietary fiber-containing substance of the present invention is contained as an active ingredient in a medicament (a pharmaceutical composition), the substance can be formulated into an IBS-inhibiting agent. The dosage form of the preparation is not particularly limited. Examples of administration routes include oral administration and enteral administration. In the case of oral or enteral administration, the substance for inhibiting IBS of the present invention can be directly administered. Alternatively, the substance in the form of solutions, suspensions, powders, granules, tablets, or capsules can be administered in combination with a pharmaceutically acceptable excipient.

Examples of the pharmaceutically acceptable excipient may include, but are not limited to, saccharides such as lactose, sucrose, and dextrose, inorganic materials such as starch, calcium carbonate, and calcium sulfate, crystalline cellulose, distilled water, purified water, and oils such as sesame oil, soybean oil, corn oil, olive oil, and cotton seed oil, which are generally used. Furthermore, other than the excipient, additives such as a binder, a lubricant, a dispersant, a suspension, an emulsifier, a diluent, a buffer, antioxidant, and a bacterium suppresser can be used. The above preparation can be mixed with another medicament or used in combination with the same. Note that, the preparation may be subjected to sterilization treatment.

A subject to which the medicament of the present invention is to be applied is not particularly limited and may be any of healthy subjects, IBS patients, patients with IBS under therapy, and healthy subjects who wish to prevent the onset of IBS, for example. Such subjects are not limited to humans and may be animals other than humans.

The dosage of the medicament of the present invention varies depending on various factors such as age, body weight, sex, and the degree of obesity of a subject; however, typically the dosage of the insoluble food-containing substance of the present invention per day is 1 g or more, 2 g or more, 3 g or more, 4 g or more, 5 g or more, 6 g or more, 7 g or more, 8 g or more, 9 g or more, and preferably 10 g or more, 12 g or more, 14 g or more, 16 g or more, 18 g or more, 20 g or more, or 30 g or more. The interval of administration is not particularly limited.

The insoluble dietary fiber-containing substance of the present invention can efficiently inhibit IBS. In addition, it is an ingredient obtained from the seeds of a grain plant via treatment with a food enzyme, so that the insoluble dietary fiber-containing substance is highly safe and is useful for use in foods or drinks or medicaments.

Furthermore, the insoluble dietary fiber-containing substance of the present invention can be produced from a relatively inexpensive material such as rice bran and wheat bran. Thus, it is excellent in view of cost performance.

When the insoluble dietary fiber-containing substance of the present invention is contained in a food or drink or a pharmaceutical composition, it can be detected by the following method. Specifically, first, the food or drink or pharmaceutical composition is suspended in water, the suspension is sieved in water using a 200-mesh sieve (ASTM standard, sieve opening: 0.075 mm), and then an insoluble fraction is recovered. Then, the insoluble fraction is stained with Nile blue or another staining reagent suitable for staining or the like of a hydrophobic substance and then observed by e.g., a fluorescent microscope, thereby detecting the insoluble dietary fiber-containing substance of the present invention. At this time, the insoluble dietary fiber-containing substance of the present invention can be distinguished from other insoluble compositions based on the property such that a layer that can be stained and a layer that cannot be stained are stacked in the insoluble dietary fiber-containing substance of the present invention. Examples of other reagents suitable for staining include Sudan III, Sudan black, oil red, and fluoro yellow 088.

### EXAMPLES

The present invention will be more specifically described by way of the following examples and test examples. However, the scope of the invention is not limited by these specific examples. In the preparation examples and test examples, unless otherwise specified, figures expressed as percentages are based on weight. Furthermore, the analytical values of ingredients were each obtained as follows. A crude protein was measured by the Kjeldahl method (and the protein conversion coefficient of nitrogen was set to 6.25) and crude lipids were quantified by Soxhlet extraction using diethyl ether as an extraction solvent. The content of a dietary fiber was obtained based on the AOAC method.

### Preparation Example 1

### Brewer's Grain→Roll Mill

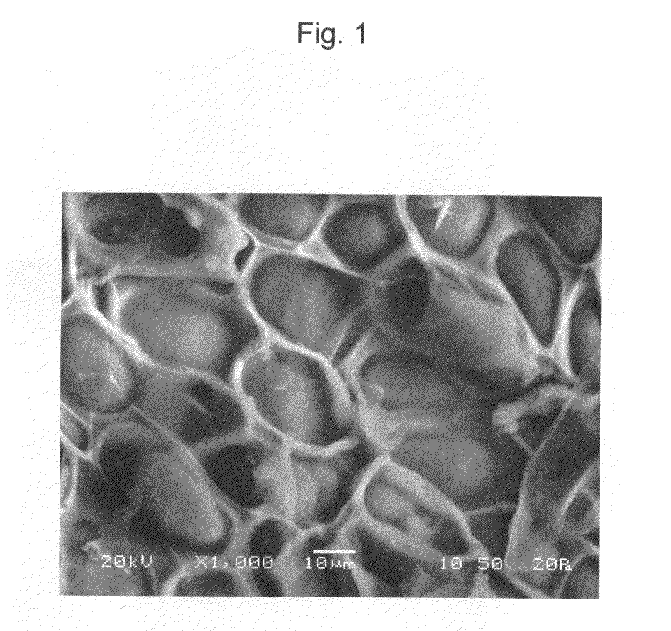

Wet brewer's grain (moisture content: 77.6 wt %) was subjected to press-peeling treatment using a roll mill (roll rotation number: 100 rpm; gap between rolls: 0.08 mm) and then sieved in water using a 16-mesh sieve (ASTM standard, sieve opening: 1.18 mm). The fraction that had passed through was further sieved using a 50-mesh sieve (ASTM standard; sieve opening: 0.300 mm). The fraction that had not passed through was recovered and subjected to lyophilization to obtain an insoluble dietary fiber-containing substance. The substance was designated as Preparation Sample 1. Analytical results are shown in Table 1 below. Furthermore, a scanning electron micrographic image of the aleurone layer portion contained in Preparation Sample 1 is shown in FIG. 1.

### Preparation Example 2

### Brewer's Grain→Roll Mill+Hemicellulase

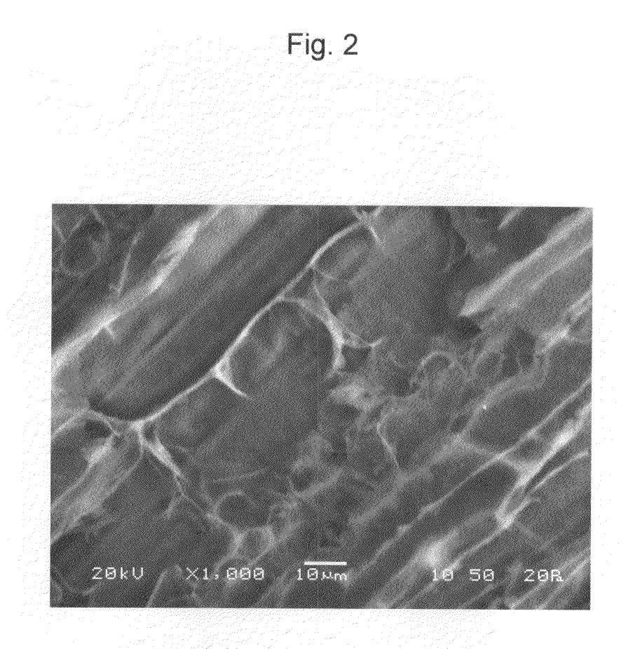

Wet brewer's grain (moisture content: 77.6 wt %) was subjected to press-peeling treatment using a roll mill (roll rotation number: 100 rpm; gap between rolls: 0.08 mm) and then sieved in water using a 16-mesh sieve (ASTM standard; sieve opening: 1.18 mm). The fraction that had passed through was further sieved using a 50-mesh sieve (ASTM standard; sieve opening: 0.300 mm). The fraction that had not passed through was recovered. A hemicellulase preparation (1.0%) (SumizymeNX, Shinnihon Chemicals Co., Ltd., Japan) was reacted overnight with the thus recovered fraction in 50 mM acetate buffer (pH 4.5; 50° C.), thereby completely degrading the aleurone layer of the plant tissue. The resultant was sieved in water using a 200-mesh sieve (sieve opening: 0.075 mm). The fraction that had not passed through was recovered and then subjected to lyophilization to obtain an insoluble dietary fiber-containing substance. The substance was designated as Preparation Sample 2. Analytical results are shown in Table 1 below. Furthermore, a scanning electron micrographic image of the aleurone layer portion contained in Preparation Sample 2 is shown in FIG. 2.

TABLE 1
Crude protein content and dietary fiber content in Preparation
Samples 1 and 2 (wt %)

| | Preparation | Preparation |
|---|---|---|
| | Sample 1 | Sample 2 |
| Crude protein | 27.1 | 11.9 |
| Dietary fiber | 60.1 | 77.6 |

### Preparation Example 3

### Naked Malt→Starch Degradation

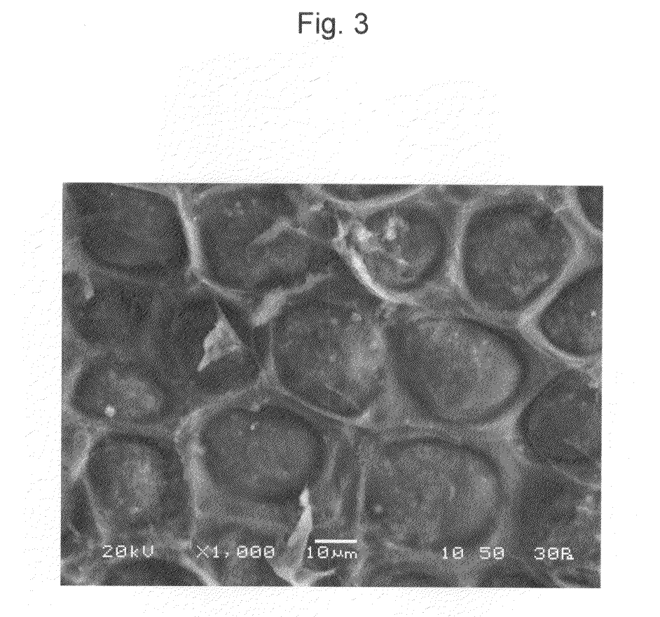

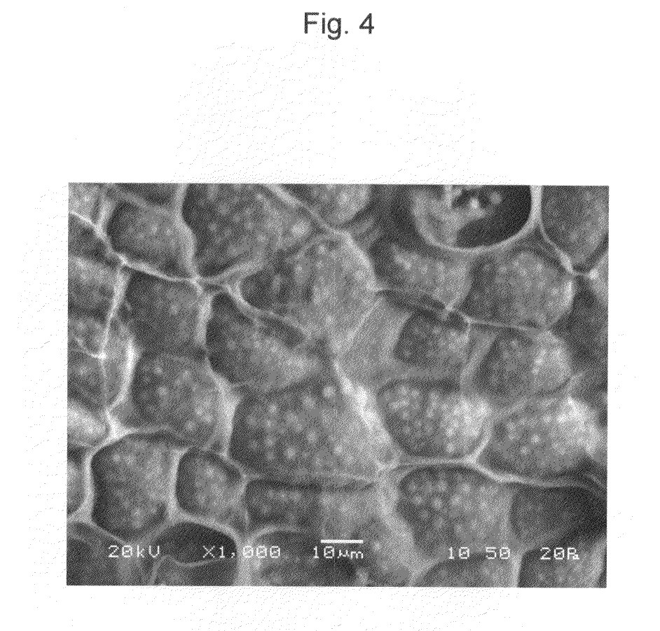

Germinated naked barley (naked malt) was used as a raw material. Husks were removed by a test rice mill TDB2A (rotation number used: 500 rpm) for brewing manufactured by SATAKE Co., Ltd., and then pulverized using a disk mill. Thereafter, water was added and the mixture was maintained at 65° C. for 3 hours to degrade starch with amylase contained in the naked malt. After starch was degraded, sieving was performed in water using a 12-mesh sieve (ASTM standard; sieve opening: 1.68 mm). The fraction that had passed through was further sieved using a 200-mesh sieve (ASTM standard, sieve opening: 0.075 mm). The fraction that had not passed through was recovered and subjected to lyophilization to obtain an insoluble dietary fiber-containing substance. The substance was designated as Preparation Sample 3. The analytical results are shown in Table 2 below. Furthermore, a scanning electron micrographic image of the aleurone layer portion contained in Preparation Sample 3 is shown in FIG. 3. Note that, when starch alone was removed with cold water without reacting with amylase at 65° C., the aleurone layer reached a state as shown in FIG. 4.

### Preparation Example 4

### Naked Malt→Starch Degradation+Roll Mill+Hemicellulase

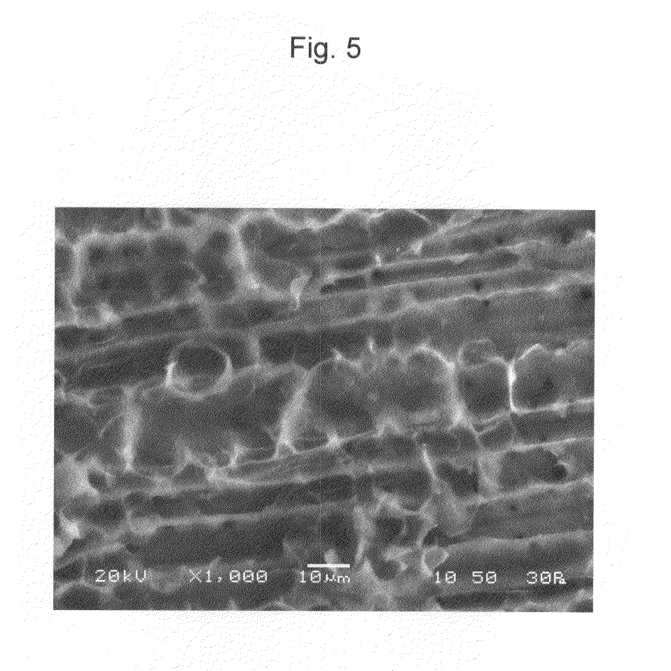

Germinated naked barley (naked malt) was used as a raw material. Husks were removed using a test rice mill TDB2A (rotation number used: 500 rpm) for brewing manufactured by SATAKE Co., Ltd., and then pulverized using a disk mill. Thereafter, water was added and the mixture was maintained at 65° C. for 3 hours to degrade starch with amylase contained in the naked malt. After starch was degraded, sieving was performed in water using a 12-mesh sieve (ASTM standard; sieve opening: 1.68 mm). The fraction that had passed through was further sieved using a 200-mesh sieve (ASTM standard; sieve opening: 0.075 mm). The fraction that had not passed through was recovered. Next, the fraction recovered in a wet state was subjected to press-peeling treatment using a roll mill (roll rotation number: 100 rpm; gap between rolls: 0.08 mm) and then sieved in water using a 200-mesh sieve (ASTM standard; sieve opening: 0.075 mm). The fraction that had not passed through was recovered and reacted overnight with a 1.0% hemicellulase preparation (Sumizyme NX, Shinnihon Chemicals Co., Ltd.) in a 50 mM acetate buffer (pH 4.5, 50° C.) to completely degrade the aleurone layer of the plant tissue. Again, sieving was performed in water using a 200-mesh sieve (ASTM standard; sieve opening: 0.075 mm). The fraction that had not passed through was recovered and subjected to lyophilization to obtain an insoluble dietary fiber-containing substance. The substance was designated as Preparation Sample 4. Analytical results are as shown in Table 2 below. Furthermore, a scanning electron micrographic image of the aleurone layer portion contained in Preparation Sample 4 is shown in FIG. 5.

TABLE 2
Crude protein content and dietary fiber content in Preparation
Samples 3 and 4 (wt %)

| | Preparation | Preparation |
|---|---|---|
| | Sample 3 | Sample 4 |
| Crude protein | 15.9 | 8.3 |
| Dietary fiber | 66.0 | 74.2 |

### Preparation Example 5

### Naked Barley→Starch Degradation

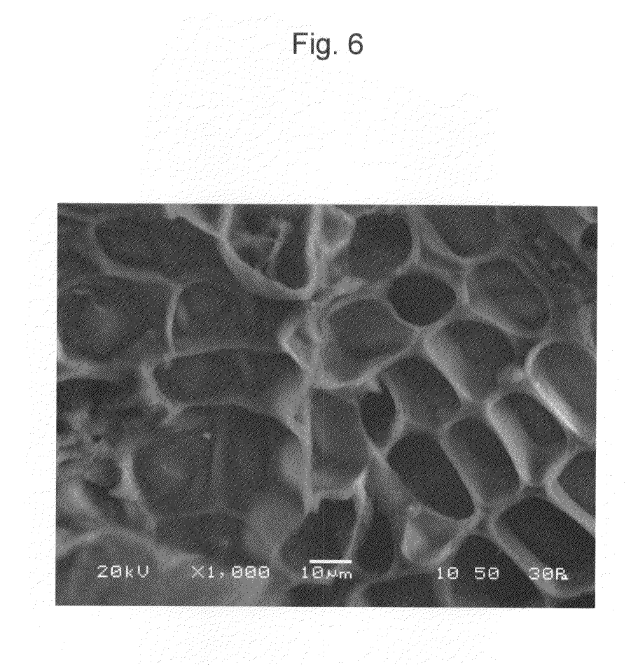

Ungerminated naked barley (naked barley) was used as a raw material. Husks were removed using a test rice mill TDB2A (rotation number used: 800 rpm) for brewing manufactured by SATAKE Co., Ltd., and then the resultant was pulverized using a disc mill. Thereafter, a 2.0% amylase preparation (Sumizyme AS, Shin Nihon Chemical Co., Ltd.) was reacted in a 50 mM acetate buffer (pH 4.5, 65° C.) for 3 hours to degrade starch in the naked barley. After starch had been degraded, sieving was performed in water using a 12-mesh sieve (ASTM standard; sieve opening: 1.68 mm). The fraction that had passed through was further sieved using a 200-mesh sieve (ASTM standard; sieve opening: 0.075 mm). The fraction that had not passed through was recovered and subjected to lyophilization to obtain an insoluble dietary fiber-containing substance. The substance was designated as Preparation Sample 5. Analytical results are as shown in Table 3 below. Furthermore, a scanning electron micrographic image of the aleurone layer portion contained in Preparation Sample 5 is shown in FIG. 6.

### Preparation Example 6

### Naked Barley→Starch Degradation+Roll Mill+Hemicellulase

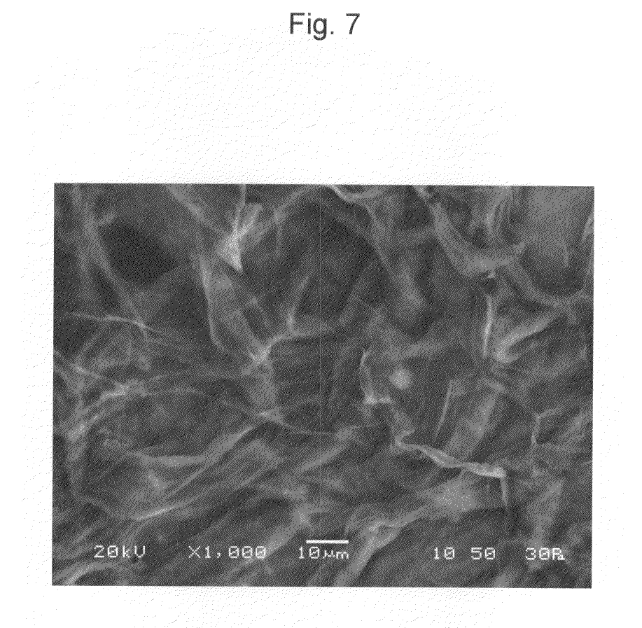

Ungerminated naked barley (naked barley) was used as a raw material. Husks were removed using a test rice mill TDB2A (rotation number used: 800 rpm) for brewing manufactured by SATAKE Co., Ltd., and then pulverized using a disc mill. Thereafter, a 2.0% amylase preparation (Sumizyme AS, Shin Nihon Chemical Co., Ltd.) was reacted in a 50 mM acetate buffer (pH 4.5, 65° C.) for 3 hours to degrade starch in the naked barley. After starch was degraded, sieving was performed in water using a 12-mesh sieve (ASTM standard; sieve opening: 1.68 mm). The fraction that had passed through was further sieved using a 200-mesh sieve (ASTM standard; sieve opening: 0.075 mm). The fraction that had not passed through was recovered. The fraction recovered in a wet state was subjected to press-peeling treatment using a roll mill (roll rotation number: 100 rpm; gap between rolls: 0.08 mm) and then sieved in water using a 200-mesh sieve (ASTM standard; sieve opening: 0.075 mm). The fraction that had not passed through was recovered. Next, the fraction recovered was reacted overnight with a 1.0% hemicellulase preparation (Sumizyme NX, Shin Nihon Chemical Co., Ltd.) in a 50 mM acetate buffer solution (pH 4.5, 50° C.) to completely degrade the aleurone layer of the plant tissue. Again, sieving was performed in water using a 200-mesh sieve (ASTM standard; sieve opening: 0.075 mm). The fraction that had not passed through was recovered and subjected to lyophilization to obtain an insoluble dietary fiber-containing substance. The substance was designated as Preparation Sample 6. Analytical results are as shown in Table 3 below. Furthermore, a scanning electron micrographic image of the aleurone layer portion contained in Preparation Sample 6 is shown in FIG. 7.

TABLE 3
Crude protein content and dietary fiber content in Preparation Samples
5 and 6 (wt %)

| | Preparation | Preparation |
|---|---|---|
| | Sample 5 | Sample 6 |
| Crude protein | 26.5 | 8.3 |
| Dietary fiber | 60.6 | 77.8 |

### Preparation Example 7

### Naked Malt→Starch Degradation+Roll Mill

Germinated naked barley (naked malt) was used as a raw material. Husks were removed using a test rice mill TDB2A (rotation number used: 500 rpm) for brewing manufactured by SATAKE Co., Ltd., and then pulverized using a disc mill. Thereafter, water was added and the mixture was maintained at 65° C. for 3 hours to degrade starch with amylase contained in the naked malt. After starch was degraded, sieving was performed in water using a 12-mesh sieve (ASTM standard; sieve opening: 1.68 mm). The fraction that had passed through was further sieved using a 200-mesh sieve (ASTM standard; sieve opening: 0.075 mm). The fraction that had not passed through was recovered. Next, the fraction recovered in a wet state was subjected to press-peeling treatment using a roll mill (roll rotation number: 100 rpm; and gap between rolls: 0.08 mm) and then sieved in water using a 200-mesh sieve (ASTM standard; sieve opening: 0.075 mm). The fraction that had not passed through was recovered and subjected to lyophilization to obtain an insoluble dietary fiber-containing substance. The substance was designated as Preparation Sample 7. Analytical results are as shown in Table 4 below.

### Preparation Example 8

### Naked Malt→Starch Degradation+Hemicellulase

Germinated naked barley (naked malt) was used as a raw material. Husks were removed using a test rice mill TDB2A (rotation number used: 500 rpm) for brewing manufactured by SATAKE Co., Ltd., and then pulverized using a disc mill. Thereafter, water was added and the mixture was maintained at 65° C. for 3 hours to degrade starch with amylase contained in the naked malt. After starch was degraded, a 1.0% hemicellulase preparation (Sumizyme NX, Shin Nihon Chemical Co., Ltd.) was reacted overnight in a 50 mM acetate buffer (pH 4.5, 50° C.) to completely degrade the aleurone layer of the plant tissue and then sieved in water using a 200-mesh sieve (ASTM standard; sieve opening: 0.075 mm). The fraction that had not passed through was recovered and subjected to lyophilization to obtain an insoluble dietary fiber-containing substance. This was designated as Preparation Sample 8. Analytical results are as shown in Table 4. below

### Preparation Example 9

### Naked Malt→Starch Degradation+Roll Mill+Hemicellulase

The same treatment as in Preparation Example 4 was performed again to obtain an insoluble dietary fiber-containing substance. The substance was designated as Preparation Sample 9. Analytical results are as shown in Table 4 below.

TABLE 4
Crude protein content and dietary fiber content in Preparation Samples
7, 8, and 9 (wt %)

| | Preparation | Preparation | Preparation |
|---|---|---|---|
| | Sample 7 | Sample 8 | Sample 9 |
| Crude protein | 12.2 | 9.6 | 8.1 |
| Dietary fiber | 71.5 | 71.5 | 72.1 |

### Preparation Example 10

### Rice Bran→Starch Degradation+Hemicellulase

Rice bran was used as a raw material. A 2.0% amylase preparation (Sumizyme AS, Shin Nihon Chemical Co., Ltd.) was reacted in a 50 mM acetate buffer (pH 4.5, 65° C.) for 3 hours to degrade starch in the rice bran. After starch was degraded, sieving was performed in water using a 200-mesh sieve (ASTM standard; sieve opening: 0.075 mm). The fraction that had not passed through was recovered and then reacted overnight with a 1.0% hemicellulase preparation (Sumizyme NX, Shin Nihon Chemical Co., Ltd.) in a 50 mM acetate buffer (pH 4.5, 50° C.) to completely degrade the aleurone layer of the plant tissue. Thereafter, sieving was performed again in water using a 200-mesh sieve (ASTM standard; sieve opening: 0.075 mm). The fraction that had not passed through was recovered and subjected to lyophilization to obtain an insoluble dietary fiber-containing substance. The substance was designated as Preparation Sample 10. Analysis results are as shown in Table 5 below.

TABLE 5
Crude protein content and dietary fiber content in Preparation
Sample 10 (wt %)

| | Preparation |
|---|---|
| | Sample 10 |
| Crude protein | 9.8 |
| Dietary fiber | 63.1 |

### Preparation Example 11

### Rice Bran→Starch Degradation+Hemicellulase

The same treatment as in Preparation Example 10 was performed again to obtain an insoluble dietary fiber-containing substance. The substance was designated as Preparation Sample 11. Analytical results are as shown in Table 6 below.

### Preparation Example 12

### Rice Bran→Starch Degradation+Hemicellulase→Defatting Treatment)

Rice bran was used as a raw material. A 2.0% amylase preparation (Sumizyme AS, Shin Nihon Chemical Co., Ltd.) was reacted in a 50 mM acetate buffer (pH 4.5, 65° C.) for 3 hours to degrade starch in the rice bran. After starch was degraded, sieving was performed in water using a 200-mesh sieve (ASTM standard; sieve opening: 0.075 mm). The fraction that had not passed through was recovered and then reacted overnight with a 1.0% hemicellulase preparation (Sumizyme NX, Shin Nihon Chemical Co., Ltd.) in a 50 mM acetate buffer (pH 4.5, 50° C.) to completely degrade the aleurone layer of the plant tissue. Thereafter, sieving was performed again in water using a 200-mesh sieve (ASTM standard; sieve opening: 0.075 mm). The fraction that had not passed through was recovered. The fraction recovered was subjected to defatting treatment with ethanol and then subjected to lyophilization to obtain an insoluble dietary fiber-containing substance. The substance was designated as Preparation Sample 12. Analytical results are as shown in Table 6 below.

TABLE 6
Crude protein content and dietary fiber content in Preparation Samples
11 and 12 (wt %)

| | Preparation | Preparation |
|---|---|---|
| | Sample 11 | Sample 12 |
| Crude protein | 9.2 | 12.6 |
| Dietary fiber | 66.0 | 78.8 |

### Preparation Example 13

### Bran→Starch Degradation+Hemicellulase

Bran was used as a raw material. A 2.0% amylase preparation (Sumizyme AS, Shin Nihon Chemical Co., Ltd.) was reacted in a 50 mM acetate buffer (pH 4.5, 65° C.) for 3 hours to degrade starch in the bran. After starch was degraded, sieving was performed in water using a 200-mesh sieve (ASTM standard; sieve opening: 0.075 mm). The fraction that had not passed through was recovered and then reacted overnight with a 1.0% hemicellulase preparation (Sumizyme NX, Shin Nihon Chemical Co., Ltd.) in a 50 mM acetate buffer (pH 4.5, 50° C.) to completely degrade the aleurone layer of the plant tissue. Thereafter, sieving was performed again in water using a 200-mesh sieve (ASTM standard; sieve opening: 0.075 mm). The fraction that had not passed through was recovered and subjected to lyophilization to obtain an insoluble dietary fiber-containing substance. This was designated as Preparation Sample 13. Analytical results are as shown in Table 7 below.

TABLE 7
Crude protein content and dietary fiber content in Preparation
Sample 13 (wt %)

| | Preparation |
|---|---|
| | Sample 13 |
| Crude protein | 4.34 |
| Dietary fiber | 88.8 |

### Preparation Example 14

### Rice Bran→Starch Degradation+Hemicellulase

Rice bran was used as a raw material. A 2.0% amylase preparation (Sumizyme AS, Shin Nihon Chemical Co., Ltd.) was reacted in a 50 mM acetate buffer (pH 4.5, 65° C.) for 3 hours to degrade starch in the rice bran. After starch was degraded, sieving was performed in water using a 200-mesh sieve (ASTM standard; sieve opening: 0.075 mm). The fraction that had not passed through was recovered and then reacted overnight with a 1.0% hemicellulase preparation (Sumizyme NX, Shin Nihon Chemical Co., Ltd.) in a 50 mM acetate buffer (pH 4.5, 50° C.) to completely degrade the aleurone layer of the plant tissue. Thereafter, sieving was performed again in water using a 200-mesh sieve (ASTM standard; sieve opening: 0.075 mm). The fraction that had not passed through was recovered and subjected to lyophilization to obtain an insoluble dietary fiber-containing substance. This was designated as Preparation Sample 14. Analytical results are as shown in Table 8 below. Furthermore, a scanning electron micrographic image of the aleurone layer portion contained in Preparation Sample 14 is shown in FIG. 8.

### Preparation Example 15

### Rice Bran→Removal of Starch by Homogenous Stirring

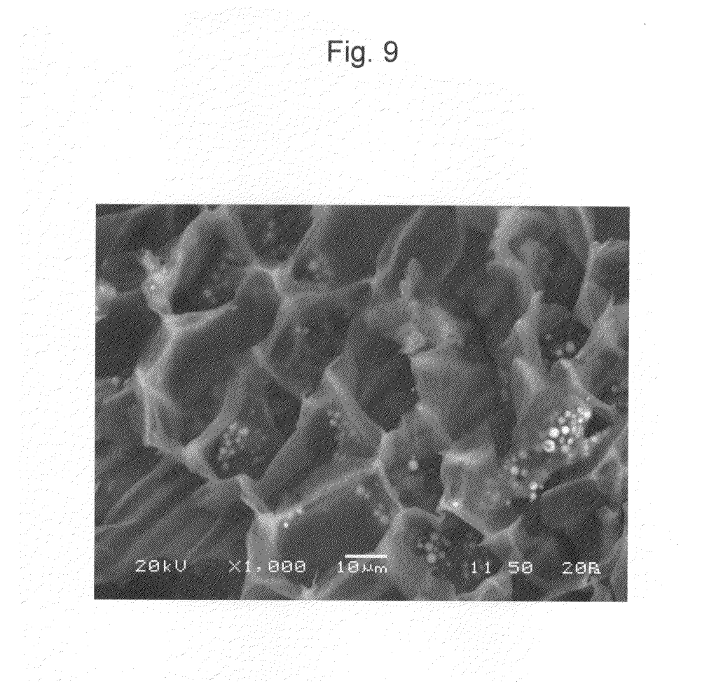

Rice bran as a raw material was suspended in water and subjected to a stirrer, T. k. robomix manufactured by Tokushu Kika Kogyo (Primix Corporation, at present) at normal temperature (10,000 RPM, about 20 minutes) and then sieved in water using a 200-mesh sieve (ASTM standard; sieve opening: 0.075 mm). The fraction that had not passed through was recovered to remove starch. This operation was repeated twice to improve the starch removal efficiency. Furthermore, during the second operation, a heat sterilization step at 80° C. was added. Finally, sieving was performed using a 200-mesh sieve (ASTM standard; sieve opening: 0.075 mm). The fraction that had not passed through was recovered and subjected to lyophilization to obtain an insoluble dietary fiber-containing substance. The substance was designated as Preparation Sample 15. Analytical results are as shown in Table 8 below. Furthermore, a scanning electron micrographic image of the aleurone layer portion contained in Preparation Sample 15 is shown in FIG. 9.

### Preparation Example 16

### Defatted Rice Bran→Starch Degradation+Hemicellulase

Defatted rice bran (Tsuno Food Industrial Co., Ltd.) was used as a raw material. A 2.0% amylase preparation (Sumizyme AS, Shin Nihon Chemical Co., Ltd.) was reacted in a 50 mM acetate buffer (pH 4.5, 65° C.) for 3 hours to degrade starch in the rice bran. After starch was degraded, sieving was performed in water using a 200-mesh sieve (ASTM standard; sieve opening: 0.075 mm). The fraction that had not passed through was recovered and then reacted overnight with a 1.0% hemicellulase preparation (Sumizyme NX, Shin Nihon Chemical Co., Ltd.) in a 50 mM acetate buffer (pH 4.5, 50° C.) to completely degrade the aleurone layer of the plant tissue. Thereafter, sieving was performed again in water using a 200-mesh sieve (ASTM standard; sieve opening: 0.075 mm). The fraction that had not passed through was recovered and subjected to lyophilization to obtain an insoluble dietary fiber-containing substance. This was designated as Preparation Sample 16. Analytical results are as shown in Table 8 below.

TABLE 8
Crude protein content and dietary fiber content in Preparation Samples
14, 15, and 16 (wt %)

| | Preparation | Preparation | Preparation |
|---|---|---|---|
| | Sample 14 | Sample 15 | Sample 16 |
| Crude protein | 12.5 | 16.0 | 19.7 |
| Dietary fiber | 59.2 | 62.8 | 71.5 |

### Preparation Example 17

### Rice Bran→Starch Degradation+Hemicellulase→Fraction that does not Pass Through 200 Mesh

Rice bran was used as a raw material. A 2.0% amylase preparation (Sumizyme AS, Shin Nihon Chemical Co., Ltd.) was reacted in a 50 mM acetate buffer (pH 4.5, 65° C.) for 3 hours to degrade starch in the rice bran. After starch was degraded, sieving was performed in water using a 200-mesh sieve (ASTM standard; sieve opening: 0.075 mm). The fraction that had not passed through was recovered and then reacted overnight with a 1.0% hemicellulase preparation (Sumizyme NX, Shin Nihon Chemical Co., Ltd.) in a 50 mM acetate buffer solution (pH 4.5, 50° C.) to completely degrade the aleurone layer of the plant tissue. Thereafter, sieving was performed again in water using a 200-mesh sieve (ASTM standard; sieve opening: 0.075 mm). The fraction that had not passed through was recovered and subjected to lyophilization to obtain an insoluble dietary fiber-containing substance. This was designated as Preparation Sample 17. Analytical results are as shown in Table 9 below.

### Preparation Example 18

### Rice Bran→Starch Degradation+Hemicellulase→200- to 500-Mesh Fraction

Rice bran was used as a raw material. A 2.0% amylase preparation (Sumizyme AS, Shin Nihon Chemical Co., Ltd.) was reacted in a 50 mM acetate buffer (pH 4.5, 65° C.) for 3 hours to degrade starch in the rice bran. After starch was degraded, sieving was performed in water using a 200-mesh sieve (ASTM standard; sieve opening: 0.075 mm). The fraction that had not passed through was recovered and then reacted overnight with a 1.0% hemicellulase preparation (Sumizyme NX, Shin Nihon Chemical Co., Ltd.) in a 50 mM acetate buffer (pH 4.5, 50° C.) to completely degrade the aleurone layer of the plant tissue. Thereafter, sieving was performed again in water using a 200-mesh sieve (ASTM standard; sieve opening: 0.075 mm). The fraction that had passed through was recovered and further sieved using a 500-mesh sieve (ASTM standard; sieve opening: 0.025 mm). The fraction that had not passed through was recovered and subjected to lyophilization to obtain an insoluble dietary fiber-containing substance. This was designated as Preparation Sample 18. Analytical results are as shown in Table 9 below.

TABLE 9
Crude protein content and dietary fiber content in Preparation Samples
17 and 18 (wt %)

| | Preparation | Preparation |
|---|---|---|
| | Sample 17 | Sample 18 |
| Crude protein | 14.2 | 12.2 |
| Dietary fiber | 76.4 | 71.6 |

### Preparation Example 19

### Rice Bran→Starch Degradation+Hemicellulase→Fraction that does not Pass Through 200 Mesh

Defatted rice bran (Tsuno Food Industrial Co., Ltd.) was used as a raw material. A 2.0% amylase preparation (Sumizyme AS, Shin Nihon Chemical Co., Ltd.) was reacted in a 50 mM acetate buffer (pH 4.5, 65° C.) for 3 hours to degrade starch in the rice bran. After starch was degraded, sieving was performed in water using a 200-mesh sieve (ASTM standard; sieve opening: 0.075 mm). The fraction that had not passed through was recovered and then reacted overnight with a 1.0% hemicellulase preparation (Sumizyme NX, Shin Nihon Chemical Co., Ltd.) in a 50 mM acetate buffer (pH 4.5, 50° C.) to completely degrade the aleurone layer of the plant tissue. Thereafter, sieving was performed again in water using a 200-mesh sieve (ASTM standard; sieve opening: 0.075 mm). The fraction that had not passed through was recovered and subjected to lyophilization to obtain an insoluble dietary fiber-containing substance. This was designated as Preparation Sample 19. Analytical results are as shown in Table 10 below.

### Preparation Example 20

### Preparation Example 19→Finely Pulverized→Fraction that Passes Through 500 Mesh

The insoluble dietary fiber-containing substance obtained in Preparation Example 19 was finely pulverized (processed by Microfoods Japan Kabushiki Kaisha), suspended in water, and then sieved in water using a 500-mesh sieve (ASTM standard; sieve opening: 0.075 mm). The fraction that had passed through was recovered and subjected to lyophilization to obtain an insoluble dietary fiber-containing substance. This was designated as Preparation Sample 20. Analytical results are as shown in Table 10 below.

TABLE 10
Crude protein content and dietary fiber content in Preparation Samples
19 and 20 (wt %)

| | Preparation | Preparation |
|---|---|---|
| | Sample 19 | Sample 20 |
| Crude protein | 13.74 | 14.72 |
| Dietary fiber | 76.5 | 75.9 |

### Test Example 1

A test was conducted using the insoluble dietary fiber-containing substance prepared according to Preparation Example 16 above in order to examine if it could improve IBS-related symptoms or parameter values.

Compositions of a control feed and an experimental feed used in the following test are as shown in Table 11.

TABLE 11
Feed composition table (unit: g/kg feed)

| | Control feed | Experimental feed |
|---|---|---|
| Casein | 146.0 | 140.4 |
| Vitamin mixture | 10.0 | 10.0 |
| Mineral mixture | 35.0 | 35.0 |
| Choline chloride | 2.0 | 2.0 |
| Cellulose | 30.0 | 0.0 |
| Insoluble dietary | | 39.3 |
| fiber-containing substance | | |
| Corn oil | 50.0 | 50.0 |
| Cornstarch | 727.0 | 723.3 |
| Total | 1000.0 | 1000.0 |

The control feed and the experimental feed were administered to experimental animals and then the animals were acclimatized. The insoluble dietary fiber-containing substance of the present invention was evaluated for its effect of inhibiting IBS using a restraint stress method (Miyata K, Kamato T, Nishida A, Ito H, Yuki H, Yamano M, Tsutsumi R, Katsuyama Y, Honda K., J Pharmacol Exp Ther. 1992; 261: 297-303.) employed for evaluation of IBS therapeutic agents for animals from among methods broadly employed for generation of IBS models. Specifically, restraint stress was added to animals using restraint cages for 4 hours a day for 3 consecutive days. The fecal output, pain threshold in the large bowel, and the serotonin content in large intestinal mucosa when the stress was added were evaluated.

### [Evaluation of Fecal Output]

In this test, ten 5-week-old male SD rats were used per group. Two groups, a control group and an experimental group, were used herein. Rats were acclimatized for 1 week to an environment and then grouped based on body weight. After grouping, rats were bred with each feed for 10 days. Five (5) rats corresponding to a half of each group were exposed to stress and the other half was not exposed to stress. After 10 days of breeding, mice were exposed to restraint stress for 3 consecutive days, wherein the duration of single exposure to stress was 4 hours and rats were exposed to restraint stress for 3 consecutive days. Exposure was specifically performed referring to the method of Miyata et al (shown before). This is briefly explained as follows. Rats were retained in restraint cages (Natsume Seisakusho Co., Ltd. (Japan)) for rats for 4 hours. Rats were then liberated and returned to general breeding cages. In addition, rats not exposed to restraint stress were kept breeding in tact.

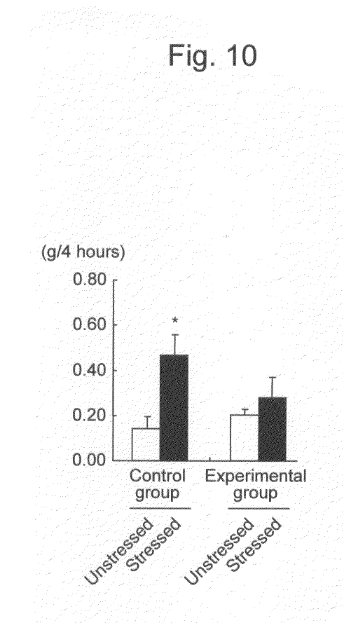

In this test, feces were sampled from all rats and dry weights thereof were measured during a period ranging from the initiation to the completion of exposure to restraint stress. An average value in 3 days was determined to be the fecal output per 4 hours. The results are shown in FIG. 10.

As shown in FIG. 10, when rats were exposed to restraint stress, the fecal output was significantly increased in the control group, but the increase in the fecal output was suppressed and anti-stress effect was confirmed in the experimental group.

### [Evaluation of Pain Threshold in Large Bowel]

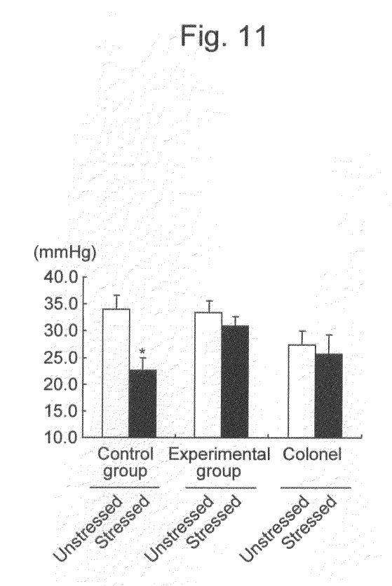

In test example 2, ten 5-week-old male SD rats were used per group and 3 groups were employed herein including a control group, an experimental group, and colonel (trade name) as positive control. After 1 week of acclimatization to an environment, rats were grouped based on body weight. Thereafter, rats were bred with each feed for 10 days. Five (5) rats corresponding to half of each group were exposed to stress and the remaining half was not exposed to stress. After 10 days of breeding, rats were exposed to restraint stress for 3 consecutive days, wherein the duration of single exposure to stress was 4 hours and rats were exposed to restraint stress for 3 consecutive days. On day 3, immediately after the completion of exposure to stress, pain thresholds were measured by a barostat method. Pain thresholds were determined based on AWR (abdominal withdrawal reflex; Al-Chaer E D, Kawasaki M, Pasricha P J., Gastroenterology. 2000 November; 119 (5): 1276-85). Pain threshold measurement was performed on another day for rats not exposed to stress. The results are shown in FIG. 11.

As shown in FIG. 11, when rats were exposed to restraint stress, a significant decrease in CRD (pain thresholds due to large bowel dilatation) was confirmed in the control group, but in the experimental group and the colonel as positive control, no such decrease occurred and a decrease in pain threshold was suppressed as confirmed in IBS.

### [Evaluation of Serotonin Content in Large Intestinal Mucosa]

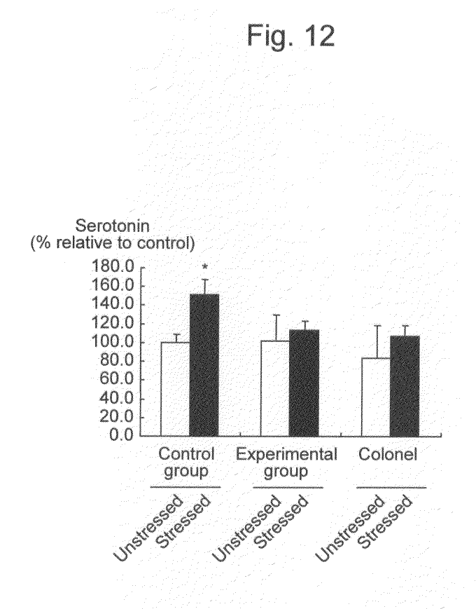

In this test, rats were bred and exposed to stress in the same manner as described above. On day 3, immediately after the completion of exposure to stress, rats were subjected to anatomy under anesthesia, large bowel was excised, and then the serotonin content in large intestinal mucosa was measured using a commercially available ELISA kit (Serotonin EIA, Labor Diagnostika Nord GmbH). The results are shown in FIG. 12.

As shown in FIG. 12, when rats were exposed to restraint stress, acute release of enterokinesis-related serotonin took place in large intestinal mucosa in the control group. A significant increase in serotonin content was observed due to exposure to stress in the control group, but in the experimental group and colonel, the increase was not significant and anti-stress effect was confirmed.

### Preparation Example 21

### Defatted Rice Bran→Starch Degradation+Hemicellulase

Defatted rice bran was used as a raw material. An insoluble dietary fiber-containing substance was prepared in a manner similar to that in Preparation Example 16. Specifically, a 1.0% amylase preparation (Sumizyme AS, Shinnihon Chemicals Co., Ltd.) was reacted for 3 hours after addition of lactic acid to adjust pH (pH 4.5, 65° C.), so as to degrade starch in the rice bran. After starch was degraded, sieving was performed in water using a 200-mesh sieve (ASTM standard; sieve opening: 0.075 mm). The fraction that had not passed through was recovered and then reacted overnight with a 0.5% hemicellulase preparation (Sumizyme NX, Shinnihon Chemicals Co., Ltd.) after addition of lactic acid to adjust pH (pH 4.5, 50° C.), so as to degrade the aleurone layer of the plant tissue. Thereafter, sieving was performed in water using a 200-mesh sieve (ASTM standard; sieve opening: 0.075 mm). The fraction that had not passed through was recovered and subjected to lyophilization to obtain an insoluble dietary fiber-containing substance. The substance was designated as Preparation Sample 21. The analytical results are shown in Table 21 below.

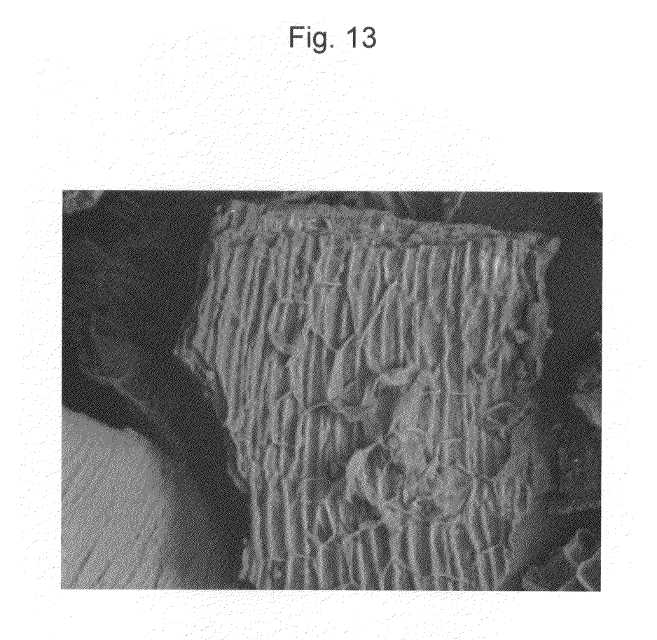

Furthermore, a scanning electron micrographic image of the aleurone layer portion contained in Preparation Sample 21 is shown in FIG. 13.

### Preparation Example 22

### Defatted Rice Bran→Starch Degradation+Hemicellulase (The Amount of Enzyme was Reduced from Preparation Example 21)

Defatted rice bran was used as a raw material. A 0.5% amylase preparation (Sumizyme AS, Shinnihon Chemicals Co., Ltd.) was reacted for 3 hours after addition of lactic acid to adjust pH (pH 4.5, 65° C.), so as to degrade starch in the rice bran. After starch was degraded, sieving was performed in water using a 200-mesh sieve (ASTM standard; sieve opening: 0.075 mm). Next, the fraction that had not passed through was recovered and then reacted overnight with a 0.2% hemicellulase preparation (Sumizyme NX, Shinnihon Chemicals Co., Ltd.) after addition of lactic acid to adjust pH (pH 4.5, 50° C.), so as to degrade the aleurone layer of the plant tissue. Sieving was performed in water using a 200-mesh sieve (ASTM standard; sieve opening: 0.075 mm). The fraction that had not passed through was recovered and subjected to lyophilization to obtain an insoluble dietary fiber-containing substance.

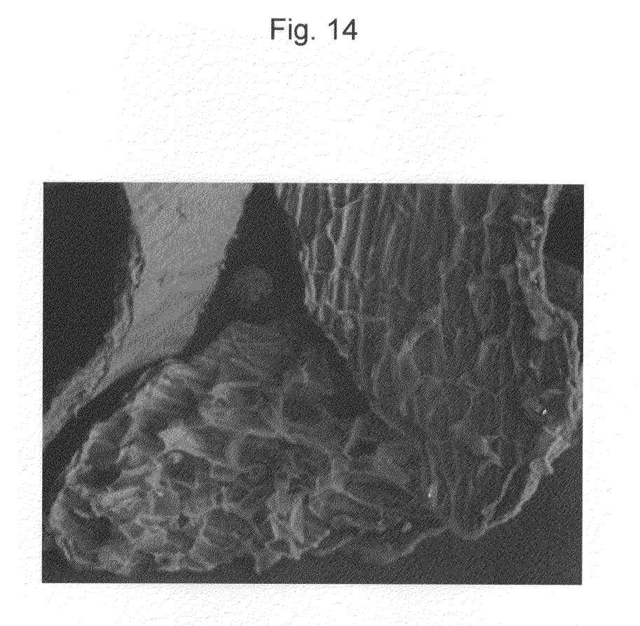

The substance was designated as Preparation Sample 22. The analytical results are shown in Table 12 below. Furthermore, a scanning electron micrographic image of the aleurone layer portion contained in Preparation Sample 22 is shown FIG. 14.

TABLE 12
Crude protein content and dietary fiber content in Preparation Samples
21 and 22 (wt %)

| | Preparation | Preparation |
|---|---|---|
| | Sample 21 | Sample 22 |
| Crude protein | 13.7 | 17.2 |
| Dietary fiber | 76.5 | 73.5 |

### Preparation Example 23

### Re-Treatment of Preparation Sample with Hemicellulase

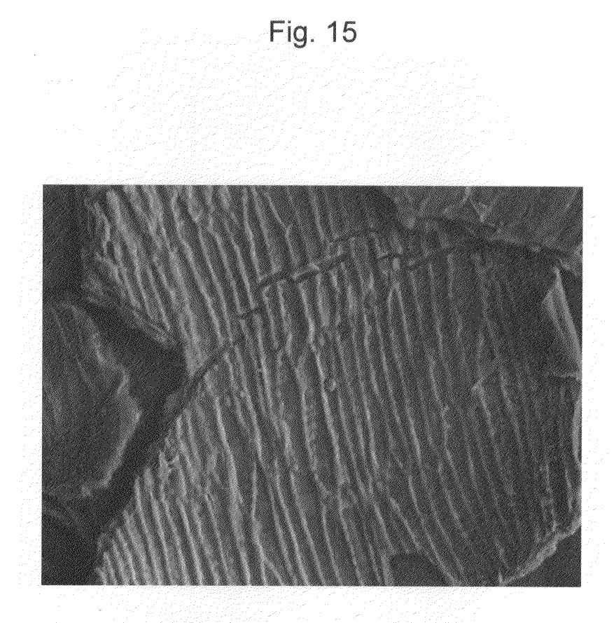

Preparation Sample 22 was treated again with a 1.0% hemicellulase preparation (Sumizyme NX, Shinnihon Chemicals Co., Ltd.) and then lactic acid was added to adjust pH (pH 4.5, 50° C.), followed by overnight reaction. Then the resultant was sieved again in water using a 200-mesh sieve (ASTM standard; sieve opening: 0.075 mm). The fraction that had not passed through was recovered and subjected to lyophilization to obtain an insoluble dietary fiber-containing substance. The substance was designated as Preparation Sample 23. The analytical results of Preparation Sample 23 are as shown in Table 13 below. Furthermore, a scanning electron micrographic image of the aleurone layer portion contained in Preparation Sample 23 is shown in FIG. 15.

TABLE 13
Crude protein content and dietary fiber content in Preparation
Sample 23 (wt %)

| | Preparation |
|---|---|
| | Sample 23 |
| Crude protein | 12.4 |
| Dietary fiber | 79.9 |

### Test Example 2

Preparation Samples 21 and 22 were treated again with a 4.0% hemicellulase preparation (Sumizyme NX, Shinnihon Chemicals Co., Ltd.), reacted overnight in 50 mM acetate buffer (pH 4.5, 50° C.), and then sieved again in water using a 200-mesh sieve (ASTM standard; sieve opening: 0.075 mm). The fraction that had not passed through was recovered and subjected to lyophilization. The recovery rate was then calculated. Furthermore, the free amounts of arabinose and xylose, which are sugars mainly constituting the aleurone layer, were also checked upon hemicellulase treatment.

### [Results of Test Example 2]

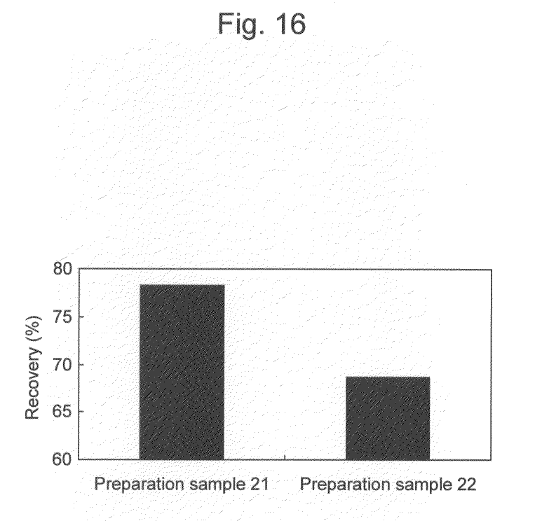

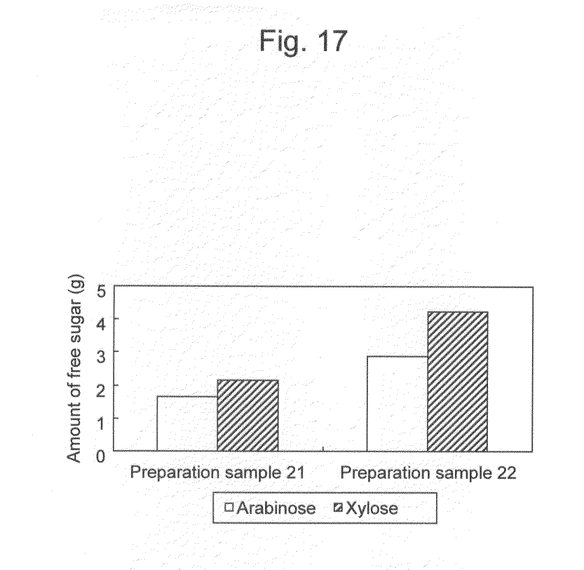

When Preparation Sample 21 was degraded again with hemicellulase, the recovery rate was 78%. When Preparation Sample 22 was similarly degraded, the recovery rate was 69% (FIG. 16). Furthermore, the amounts of free arabinose and xylose were checked when each sample was degraded. As a result, it was found that the amounts of free arabinose and xylose were both higher when Preparation Sample 22 had been degraded (FIG. 17). Arabinose and xylose are sugars mainly constituting the aleurone layer. From the fact that these sugars are liberated by hemicellulase treatment, it can be presumed that the aleurone layer was degraded by hemicellulase treatment.

Preparation Sample 21 was prepared in a manner similar to that in Preparation Example 16 by which the IBS-inhibiting effect was actually demonstrated in Test example 1. From the above results, it can be presumed that a Preparation Sample exerting a recovery rate of at least about 70% or more, and preferably about 80% or more when subjected to re-degradation via hemicellulase treatment under conditions similar to those of the relevant test examples is confirmed to have an effect of inhibiting IBS. Furthermore, the degradation degree of the aleurone layer can be expressed using an index.

### Test Example 3

### Effects of Improving Clinical Findings in Irritable Bowel Syndrome Patients

Among IBS patients who fulfilled ROME III criteria as patient selection criteria, patients subjected to the test: were diarrheal-type patients who were 16- to 74-year-old male or female patients; were outpatients whose participation in the test was determined by doctors; agreed to ingest the insoluble dietary fiber-containing substance prepared according to Preparation Example 16 above after sufficient explanation; and agreed to cooperate with the ingestion test. (Exclusion criteria: among cases with other complications, cases requiring the intake of remedies considered to have effects on drug efficacy, cases complicated by cancer or dysplasia, cases of women who might have been pregnant, cases of pregnant women, cases involving previous gastrointestinal tract resection, cases regarding which ileus symptoms had been confirmed, cases involving allergic symptoms against rice, and other cases in which subjects were designated by doctors as inappropriate as subjects for the ingestion test.) The thus selected patients ingested 10 g to 15 g of the insoluble dietary fiber-containing substance per day for 4 weeks. After ingestion, clinical findings, patients' impressions concerning life, and the like were evaluated based on the following parameters. Test participants were four men and one woman (with an average age of 31 years old). All of them had ingested IBS therapeutic agents and had experienced resistance. The evaluation method was performed as shown in Table 14 according to Am J Gastroenterol 101; 1581-1590, 2006.

TABLE 14

| | |
|---|---|
| Evaluation parameters | Stage assessment |
| Defecation frequency | Frequency/day |
| Stool properties | Bristol Stool Scale of 1 to 7 |
| Abdominal pain/abdominal discomfort | Scale of 1 to 6 |
| Feeling of abdominal bloating/bloating | Scale of 1 to 6 |
| Urgent need to defecate | Scale of 1 to 6 |
| Sense of incomplete evacuation | Scale of 1 to 6 |
| Straining | Scale of 1 to 6 |
| Passage of gas and mucus | Scale of 1 to 6 |
| Satisfaction concerning defecation | Scale of 1 to 6 |
| Overall improvement | Scale of 1 to 6 |

Determination was made for each parameter using a scale of 1 to 6:

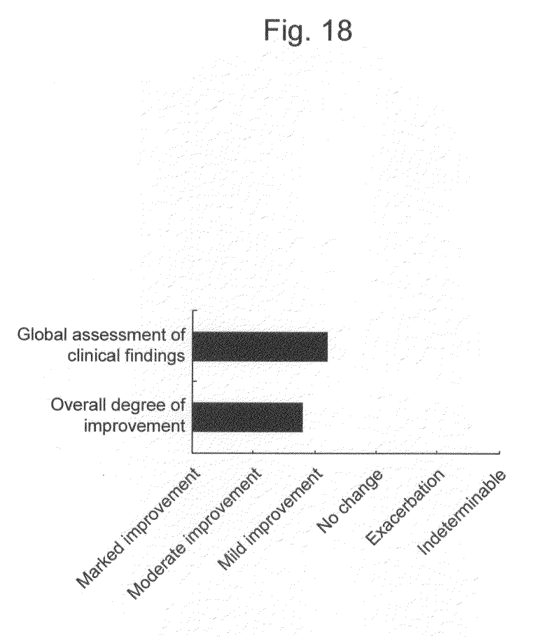

1: marked improvement; 2: moderate improvement; 3: mild improvement; 4: no change; 5: exacerbation; and 6: indeterminable. Also, in addition to the above indices, regarding global assessment of clinical findings, a doctor in charge of the test made similar evaluations using a scale of 1 to 6 in view of the overall results concerning safety. The results are shown in FIG. 18.

As shown in FIG. 18, it was confirmed that overall degree of improvement had increased and global assessments of clinical findings had improved through ingestion of the insoluble dietary fiber-containing substance for 4 consecutive weeks (average dose of 12 g/day). Furthermore, in this test, no adverse event correlated with the ingestion of the insoluble dietary fiber-containing substance was observed. It was considered that the insoluble dietary fiber-containing substance posed no safety concerns for patients.

### INDUSTRIAL APPLICABILITY

According to the present invention, a novel substance for inhibiting IBS (hereinafter, also referred to as an IBS-inhibiting substance) is provided.

The IBS-inhibiting substance of the present invention has advantages such that: it is rich in insoluble dietary fiber from the seeds of a grain plant and preferably a Gramineae plant; and no adverse reaction takes place. The substance is useful for use in a food or drink or medicament for inhibiting IBS.

The substance of the present invention containing insoluble dietary fiber from the seeds of a grain plant and preferably a Gramineae plant is provided as an IBS-inhibiting substance capable of exerting the effect of inhibiting IBS such that the substance can effectively alleviate one or more IBS-related symptoms such as acute excretion of feces or decreased pain threshold upon exposure to stress.

All publications, patents, and patent applications cited herein are incorporated herein by reference in their entirety.

1. A substance for inhibiting irritable bowel syndrome, containing insoluble dietary fiber from the seeds of a grain plant, wherein the substance is produced by treatment comprising the steps of:
(a) preparing a raw material by pulverizing or polishing the seeds of a grain plant and then recovering the resultant outer fraction of the seeds;
(b) subjecting the raw material to starch removal treatment to prepare a starch-free fraction;
(c) enzymatically treating the fraction prepared in step (b) with an enzyme having hemicellulase activity; and
(d) recovering an insoluble fraction from the enzymatically treated solution.

2. The substance according to claim 1, wherein the grain plant is a Gramineae plant.

3. The substance according to claim 2, wherein the Gramineae plant is rice, barley, rye, or wheat.

4. The substance according to claim 1, wherein in step (b), the starch removal treatment is performed by enzymatic treatment using amylase or glucoamylase.

5. The substance according to claim 1, wherein in step (b), the starch removal treatment is performed by heat gelatinization treatment.

6. The substance according to claim 1, wherein in step (b), the starch removal treatment is performed by physical destruction treatment.

7. The substance according to claim 6, wherein the physical destruction treatment is performed using a homogenizer.

8. The substance according to claim 1, wherein in step (b), the starch-free fraction is further subjected to press-peeling treatment.

9. The substance according to claim 1, wherein the raw material to be subjected to the starch removal treatment is rice bran, wheat malt, or barley malt.

10. The substance according to claim 1, wherein the raw material to be subjected to the starch removal treatment is defatted rice bran.

11. The substance according to claim 8, wherein the raw material to be subjected to the starch removal treatment is wheat bran or polished-barley residue.

12. The substance according to claim 8, wherein the starch-free fraction is brewer's grain.

13. The substance according to any one of claims 1 to 12, wherein the enzyme having hemicellulase activity is xylanase.

14. The substance according to any one of claims 1 to 13, wherein, in step (c), the enzyme is used in combination with protease.

15. The substance according to any one of claims 1 to 14, wherein step (c) is further followed by defatting treatment.

16. The substance according to any one of claims 1 to 15, wherein the insoluble fraction in step (d) comprises a fraction having a grain size that allows it to substantially pass through a 5- to 25-mesh ASTM standard sieve (American Society for Testing and Materials) but not to pass through a 500-mesh ASTM standard sieve.

17. The substance according to claim 16, wherein the insoluble fraction in step (d) comprises a fraction having a grain size that does not substantially allow it to pass through a 200-mesh ASTM standard sieve.

18. The substance according to any one of claims 1 to 17, wherein the protein content is 20 wt % or less and the dietary fiber content is 55 wt % or more.

19. The substance according to any one of claims 1 to 18, wherein an aleurone layer is partially or completely removed from the insoluble dietary fiber.

20. A substance for inhibiting irritable bowel syndrome containing an insoluble dietary fiber from the seeds of a grain plant, having the following properties:
(i) the substance comprises a fraction having a grain size that allows it to substantially pass through a 5- to 25-mesh ASTM standard sieve (American Society for Testing and Materials) but not to pass through a 500-mesh ASTM standard sieve;
(ii) the protein content is 20 wt % or less and the dietary fiber content is 55 wt % or more; and
(iii) the aleurone layer of insoluble dietary fiber is partially or completely removed.

21. The substance according to claim 20, comprising a fraction that does not substantially pass through a 200-mesh ASTM standard sieve.

22. A food or drink for inhibiting irritable bowel syndrome, containing the substance according to any one of claims 1 to 21 as an active ingredient.

23. A pharmaceutical composition for inhibiting irritable bowel syndrome, containing the substance according to any one of claims 1 to 21 as an active ingredient.

24. Use of the substance according to any one of claims 1 to 21 for producing a food or drink or medicament for inhibiting irritable bowel syndrome.

25. A method for producing a substance for inhibiting irritable bowel syndrome containing insoluble dietary fiber from the seeds of a grain plant, comprising the following steps of:
(a) preparing a raw material by pulverizing or polishing the seeds of a grain plant and then recovering the resultant outer fraction of the seeds;
(b) subjecting the raw material to starch removal treatment to prepare a starch-free fraction;
(c) enzymatically treating the fraction prepared in step (b) with an enzyme having hemicellulase activity; and
(d) recovering an insoluble fraction from the enzymatically treated solution.

26. A method for producing the substance according to claim 20, comprising the following steps of:
(a1) preparing a raw material by pulverizing or polishing the seeds of a grain plant and then recovering the resultant outer fraction of the seeds;
(b1) subjecting the raw material to starch removal treatment via physical destruction to prepare a starch-free fraction; and
(c1) recovering, from the fraction prepared in step (b1), an insoluble fraction that passes through a 5- to 25-mesh ASTM standard sieve (American Society for Testing and Materials), but does not pass through a 500-mesh ASTM standard sieve.

27. The substance for inhibiting irritable bowel syndrome according to claim 20 or 21, which is produced by the method according to claim 25 or 26.
